(19) United States
(12) Patent Application Publication
(10) Pub. No.: US 2021/0368750 A1
(43) Pub. Date: Dec. 2, 2021

(54) BOS TAURUS CELL TYPE 'HO840003210132823'

(71) Applicant: ABS Global, Inc., DeForest, WI (US)

(72) Inventors: Katrina Dattilo, Waunakee, WI (US); Devan Funk, DeForest, WI (US)

(73) Assignee: ABS Global, Inc., DeForest, WI (US)

(21) Appl. No.: 17/323,658

(22) Filed: May 18, 2021

Related U.S. Application Data
(60) Provisional application No. 63/031,761, filed on May 29, 2020.

(51) Int. Cl. A01K 67/027; A01N 1/02; C12N 5/073; C12N 5/075; C12N 5/0735

(52) U.S. Cl. CPC A01K 67/027; A01N 1/0221; A01K 2227/101; C12N 5/0609; C12N 5/0611; C12N 5/0604

(57) ABSTRACT

The disclosure relates to Bovine germplasm of Bos taurus HO840003210132823. Included in the present disclosure are cells comprising the genome of Bovine HO840003210132823 characterized by the presence of homozygous loci and spermatozoa obtained from said cells. Also provided by the present disclosure are tissue cultures of cells, animals obtained from said cells, and parts thereof, including F1 spermatozoa. The disclosure further provides for methods of breeding, selecting, and using the germplasm to improve existing commercial cattle herds generated from in vitro fertilization methods and progeny cattle obtained from in vitro fertilization and implantation and artificial insemination methods.

### CROSS REFERENCE TO RELATED APPLICATION

This application claims the benefit of and priority to U.S. Provisional Application 63/031,761, filed on May 29, 2020. 63/031,761 is hereby incorporated by reference in its entirety.

### INCORPORATION OF SEQUENCE LISTING

The Sequence Listing, including the file named DG-66-2020-US1-SEQLST.txt, which is 12,898,823 bytes in size, was created on May 18, 2021 and is hereby incorporated by reference in its entirety.

### FIELD

The present disclosure relates to the field of Bos taurus breeding. In particular, the present disclosure related to Bos taurus HO840003210132823 having high multi-trait selection indices and high trait transmissibility.

### BACKGROUND

There are numerous steps in the development of any new, desirable Bos taurus germplasm. Bos taurus breeding begins with the analysis and definition of problems and weaknesses of the current germplasm, the establishment of program goals, and the definition of specific breeding objectives. The next step is selection of germplasm that possesses the traits to meet the program goals. A goal is to combine in a single animal an improved combination of desirable traits from the parental germplasm. See Schefers et al., Animal Frontiers 2(1):1-9 (2012).

During breeding, cattle breeders have a variety of sources when making breeding decisions. In addition to genomic data, a number of agencies and organizations collect and release analysis of population data and indexes. Every three months, the Animal Improvement Programs Laboratory (AIPL) of the United States Department of Agriculture releases the newest USDA-DHIA (Dairy Herd Improvement Association) genetic evaluations for dairy bulls and cows. The AIPL calculates genetic evaluations for type for various breeds, and many breed associations provide their own indexes or other strategies for evaluating certain breed-relevant traits. U.S. dairy genetic evaluations are computed every four months by the Council on Dairy Cattle Breeding (CDCB) and Holstein Association USA (HAU). Both CDCB and HAU traits provide the breeder with important comparative data to evaluate the complex genetic and phenotypic traits to develop improved and desirable Bos taurus germplasm. For Holstein and Jersey sires, for example, evaluations are genomically enhanced and represent a blending of genomic data, pedigree information, and results from progeny. These genetic evaluations provide the breeder with important information for the selection of desirable germplasm and the development of new and valuable inseminates.

There is a continuous need to develop improved Bos taurus germplasm for use in improving production herds as well as for the continued improvement of elite animals.

### SUMMARY

The present disclosure provides for and includes elite Bos taurus germplasm. In various embodiments, the present teachings can provide for a Bos taurus cell of type Animal, a representative sample of cells of type Animal comprising the Deposit. In some configurations, the present teachings can include a frozen vial, a cell culture, a tissue, a zygote, or an embryo comprising a plurality of the Bos taurus cell disclosed herein. In various configurations, the present teachings can include a bull comprising a plurality of the Bos taurus cell disclosed herein. In various configurations, the cell can be a sperm cell. In some configurations, semen can comprise a plurality of the sperm cell of the present teachings.

In various configurations, the present teachings can include an embryo produced by contacting the cell with a Bos taurus ovum.

In various configurations, the present teachings can include a composition of matter comprising the cell and a Bos taurus ovum.

In various embodiments, the present teachings can provide for a Bos taurus cell produced by somatic cell nuclear transfer of a Bos taurus cell of type Animal, a representative sample of cells of type Animal comprising the Deposit. In some configurations, the present teachings can include a frozen vial, a cell culture, a tissue, a zygote, or an embryo comprising a plurality of the Bos taurus cell. In various configurations, the present teachings can include a bull comprising a plurality of the Bos taurus cell. In various configurations, the cell can be a sperm cell. In some configurations, semen can comprise the sperm cell of the present teachings.

In various embodiments, the present teachings can provide for a Bos taurus cell from an F1 offspring of an animal comprising a cell of type Animal, a representative sample of cells of type Animal comprising the Deposit. In some configurations, the present teachings can include a frozen vial, a cell culture, a tissue, a zygote, or an embryo comprising a plurality of the Bos taurus cell from an F1 offspring. In various configurations an animal can comprise a plurality of the Bos taurus cell from the F1 offspring. In some configurations, the present teachings can include a container of semen produced by the animal wherein the animal is a bull. In some configurations, the animal can be a bull, a cow, or a heifer. In various configurations, the animal can be a bull. In various configurations, the animal can be a cow or heifer. In various configurations, the F1 offspring can be a gene edited animal.

In various configurations, the cell can be an ovum.

In various configurations, the cell can be a sperm cell. In some configurations, the present teachings can include semen comprising a plurality of the Bos taurus sperm cell.

In some various configurations, the present teachings can include an embryo produced using a gamete of the F1 animal of the present teachings. In various configurations, the present teachings can include an embryo produced by contacting the sperm cell of the F1 bull with a Bos taurus ovum. In various configurations, the present teachings can include an embryo produced by contacting an ovum of the F1 cow or heifer of the present teachings with Bos taurus sperm cell.

In various configurations, the present teachings can include a composition of matter comprising a gamete of the F1 animal of the present teachings and a gamete cell from a second Bos taurus parent. In various configurations, the present teachings can include a composition of matter comprising the F1 sperm cell of the present teachings and a Bos taurus ovum. In various configurations, the present teachings can include a composition of matter comprising the F1 ovum of the present teachings and a Bos taurus sperm cell. The present disclosure provides for and includes elite Bos taurus germplasm. In various embodiments of the present teachings can provide for a Bos taurus cell having a genome comprising at least 90% of loci comprising a Homozygous Genotype of Animal, a representative sample of which comprises the Deposit. In some configurations, the genome comprises 95% of loci comprising the genotype of Animal.

In various configurations, the Bos taurus cell can further comprise a plurality of the cell comprising a frozen vial of the plurality of the cell, a cell culture, a tissue, a zygote, an embryo, a calf, or a mature adult. In various configurations, the Bos taurus cell further comprises a plurality of the cell comprising a bull.

In various embodiments, the present disclosure provides for and includes a Bos taurus cell comprising a genome comprising at least 90% of loci comprising a Haploid Genotype of Animal, a representative sample of which comprises the Deposit. In some configurations, the genome comprises at least 95% of loci comprising a Haploid Genotype of Animal. In various configurations, the cell is a gamete. In various configurations, the cell is a sperm. In some configurations, the sperm further comprises a plurality of sperm comprising semen. In some configurations, the semen is contained in a straw. In some configurations, the straw of semen is cryopreserved.

In various embodiments, the present disclosure provides for and includes a Bos taurus cell from an F1 offspring of an animal having a genome comprising at least 90% of loci comprising the Homozygous Genotype of Animal, a representative sample of Animal comprises the Deposit. In some configurations, the F1 offspring cell comprises at least 70% of loci that comprise a Homozygous Genotype of Animal. In various configurations, the at least 70% of loci that comprise a Homozygous Genotype of Animal is at least 80% of loci that comprise a Homozygous Genotype of Animal. In various configurations, the at least 70% of loci that comprise a Homozygous Genotype of Animal is at least 90% of loci that comprise a Homozygous Genotype of Animal. In various configurations, the at least 70% of loci that comprise a Homozygous Genotype of Animal is at least 95% of loci that comprise a Homozygous Genotype of Animal. In various configurations, the at least 70% of loci that comprise a Homozygous Genotype of Animal is at least 98% of loci that comprise a Homozygous Genotype of Animal. In various configurations, the F1 offspring further comprises at least one gene edited sequence. In various configurations, the cell from an F1 offspring further comprises a plurality of the cell comprising meat. In various configurations, the cell from an F1 offspring further comprises a plurality of the cell comprising a frozen vial of the plurality of the cell, a cell culture, a tissue, a zygote, an embryo, a calf, or a mature adult. In various configurations, the cell from an F1 offspring further comprises a plurality of the cell comprising a bull.

In various embodiments, the present disclosure provides for and includes a Bos taurus haploid cell from an F1 offspring of an animal having a genome comprising at least 90% of loci comprising the Homozygous Genotype of Animal, a representative sample of Animal comprising the Deposit. In various configurations, the haploid cell comprises at least 50% of loci that comprise a Homozygous Genotype of Animal. In various configurations, the haploid cell comprises at least 66% of loci that comprise a Homozygous Genotype of Animal. In various configurations, the haploid cell comprises at least 75% of loci that comprise a Homozygous Genotype of Animal. In various configurations, the haploid cell comprises at least one gene edited sequence. In various configurations, the cell is a gamete. In various configurations, the cell is an ovum. In various configurations, the cell is a sperm. In some configurations, the sperm further comprises a plurality of sperm comprising semen. In various configurations, the semen is contained in a straw. In various configurations, the straw of semen is cryopreserved.

In various embodiments the present disclosure provides for and includes a method of producing an embryo comprising crossing Animal to a cow or heifer. In some configurations the embryo can be a cow embryo. In various configurations, the embryo can be a calf embryo. In various configurations, the present disclosure provides for and includes an F1 offspring or part thereof of Animal that matured from an embryo produced by the instant method. In various configurations, the part of the F1 offspring can be a gamete. In various configurations, the gamete can be a sperm.

### DETAILED DESCRIPTION

A goal of a Bos taurus breeding program is to combine in a single Bos taurus animal an improved combination of desirable traits from the parental germplasm that provides for desirable progeny when used in artificial insemination programs, in vitro fertilization programs, Embryo transfer programs, or a combination thereof. Improved Bos taurus inseminate varieties are useful for various artificial breeding techniques, including artificial insemination (“AI”) and embryo transfer (“ET”). Improved Bos taurus germplasm, varieties, oocytes, embryos, and inseminates prepared therefrom, are desirable.

The present disclosure provides for, and includes, an improved elite germplasm obtained from a multigenerational breeding program. The germplasm is unique and readily distinguishable from germplasm present in non-selected cattle. Indeed, in the absence of continued selection, the germplasm reverts to heterogeneity and diversity. As provided herein, the germplasm of the present disclosure is identifiable using standard methods and the germplasm can be readily identified in progeny generations. Indeed, as few as 800 SNP markers are sufficient to identify parentage with greater than 99% accuracy. See McClure et al., Frontiers in Genetics 9(84):1-14 (2018). As provided here, the tens of thousands of sequences provide for tracking and selecting animals through multiple generations. Breeding with the germplasm provided herein, combined with the selection of suitable mates, will maintain the desirable germplasm in subsequent generations. Moreover, genetic testing allows for the removal of progeny having germplasm that lacks that set of desired loci for the improvement of cattle herds.

It is to be understood that the disclosure is not necessarily limited in its application to the details set forth in the following description or exemplified by the Examples. The disclosure is capable of other aspects or of being practiced or carried out in various ways.

### Definitions

“Animal” as used herein, refers to cells, animals, or particularly gametes and embryos of Bos taurus HO840003210132823. The terms “Animal” and “HO840003210132823” are used interchangeably without any change in their meaning.

“Diploid Genotype” as used herein refers to a genotype containing two copies of each chromosome. This genotype is generally present in somatic cells.

“Haploid Genotype” as used herein, refers to a genotype containing one copy of each chromosome. Therefore, relative to a diploid genotype for a given individual, it will contain half of the loci comprising a diploid chromosome. This term can refer to the genotype of a haploid cell, or to one half of the genotype of a diploid cell. This genotype is generally present in gametes.

“Homozygous Genotype” as used herein refers to the loci of a diploid genome wherein both chromosomes have the same allele at a given locus.

“Heterozygous Genotype” as used herein, refers to the loci of a diploid genome wherein each chromosome has a different allele at a given locus.

“Animal Genotype” as used herein, refers to the genotype of Animal and is characterized by the SNPs and flanking regions recited in SEQ ID NOs: 1 to 41546.

“Animal Homozygous Genotype” as used herein, refers to the loci in Animal that are homozygous and is characterized by the SNPs and flanking regions recited in SEQ ID NOs: 1 to 28548.

“Animal Heterozygous Genotype” as used herein, refers to the Heterozygous Genotype of Animal and is characterized by the SNPs and flanking regions recited in SEQ ID NOs: 28549 to 41546.

“Animal Haploid Genotype”, as used herein, refers to a Haploid Genotype derived from Animal and comprises at least one copy of the SNPs and flanking regions of the Animal Homozygous Genotype and, on average, at least one copy of 50% of the SNPs and flanking regions of the Animal Heterozygous genotype. This can refer to the genotype of a haploid cell, or to one half of the genotype of a diploid cell.

“Animal F1 Genotype,” as used herein, refers to the genome of an F1 offspring derived from Animal and a second parent. This offspring has a set of chromosomes comprising a pair of chromosomes for each chromosome (1-29 and a sex chromosome): one member of the pair inherited from Animal, the other member of the pair inherited from the second parent.

“Deposit” as used herein, refers to a representative sample of cells of Animal deposited under ATCC Accession No. TBD on TBD. The Deposit contains cells obtained from Animal as described in Example 3. The Deposit was made under the Budapest Treaty with the American Type Culture Collection (ATCC), 10801 University Boulevard, Manassas, Va. 20110-2209 USA. Upon issuance, all restrictions on the availability to the public of the deposit will be irrevocably removed consistent with all of the requirements of the Budapest Treaty and 37 C.F.R. §§ 1.801-1.809. Applicant does not waive any infringement of rights granted under this patent.

The term “Cell of Type Animal” means any type of cell having the same genotype across each of the nuclear chromosomes as the cell line comprising the Deposit. As used herein, two cells may have the same genotype even when read errors or mutations make the measured DNA sequences of the two cells not completely identical. While the cell line comprising the Deposit is a diploid line, a plurality of haploid Cells of Type Animal can be genotyped and can have the same genotype (allowing for read errors or mutations) as the cells comprising the Deposit. Multiple haploid Cells of Type Animal have the same average genotype as the cell line comprising the Deposit. For any given cell, meiotic recombination may result in chromosomes that have a different arrangement of alleles than the parent, but a plurality of haploid cells from an animal with an Animal cell type can be genotyped and will have the same genotype as the cells comprising the Deposit. The germplasm of Animal inherently comprises this genotype, and therefore can be defined by a cell of type Animal.

The phrase “producing an embryo from an animal” as used herein refers to the process of fertilization. With regard to the bull, this refers to contact of the bull's sperm cells with a Bos taurus ovum resulting in fertilization of the ovum to produce an embryo. Regarding the cow or heifer, this refers to contact of an ovum of the cow or heifer with Bos taurus sperm cells. Contacting a sperm and an ovum can comprise natural service, artificial insemination, in vitro fertilization or any other method wherein an ovum and sperm are brought into proximity such that they can join to form an embryo.

“Sire” as used herein, refers to the sire of Animal, Bos taurus HO840003141494670.

“Dam” as used herein, refers to the dam of Animal, Bos taurus HO840003143328234.

“Paternal Grand-Sire” as used herein, refers to the paternal grand sire of Animal, Bos taurus HO840003129038181.

“Paternal Grand-Dam” as used herein, refers to the paternal grand dam of Animal, Bos taurus HO840000074258448.

“Maternal Grand Sire” as used herein, refers to the maternal grand sire of Animal, Bos taurus HO840003128557482.

“Maternal Grand Dam” as used herein, refers to the paternal grand dam of Animal, Bos taurus HO840003133318719.

Offspring, as used herein, refers to the progeny of an animal created through natural service, artificial insemination, somatic cell nuclear transfer, or in vitro fertilization (IVF).

As used herein, “germplasm” includes intact genomes comprising chromosomes present in cells or nuclei. The term “germplasm” may include any gamete or germ cell, or any somatic cell from which an animal can be cloned.

As used herein the term “about” refers to ±10%.

As used herein, the singular forms “a,” “an,” and “the” include plural references unless the context clearly dictates otherwise. For example, the term “a compound” or “at least one compound” may include a plurality of compounds, including mixtures thereof.

Throughout this application, various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3,” “from 1 to 4,” “from 1 to 5,” “from 2 to 4,” “from 2 to 6,” “from 3 to 6,” etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

For the purposes of this disclosure, the term “semen” means seminal fluid which may contain sperm (also referred to as “spermatozoa”) secreted by the gonads of a male animal which can be collected from the male animal by any method known to those in the animal breeding arts.

As used herein, “locus”, or plural “loci”, refers to a physical site or location of a specific gene or marker on a chromosome. Loci of the present disclosure include, and are identifiable by, a SEQ ID NO, each SEQ ID NO providing the identity of the polymorphism at a single nucleotide polymorphic (SNP) site and the adjacent 100 base pairs.

Loci may also be characterized as either ‘A’ loci, ‘B’ loci, or heterozygous ‘A/B’ loci. The identification of loci as either ‘A’ or ‘B’ loci is determined according to the top (TOP) and bottom (BOT) designations based on the polymorphism itself, or the contextual surrounding sequence as developed by ILLUMINA®, Inc. (San Diego, Calif.). Methods for determining the designation of a polymorphic site as ‘A’ or ‘B’ are known in the art, for example as provided by ILLUMINA®'s Technical Note entitled ““TOP/BOT” Strand and ‘A/B’ Allele”, available on the internet at www(dot)illumina(dot)com/documents/products/technotes/technote_topbot.pdf. The NCBI's dbSNP database adopted the TOP/BOT nomenclature in 2005 and the designation is well known to those of skill in the art. As shown in the Examples, thorough sequence comparison extensive information about each locus is available to a person of ordinary skill in the art, including, but not limited to, dbSNP identifier, sources, chromosomal location, genes, transcripts, linkage to genes or quantitative trait loci (QTL), and interactions.

In diploid cells, the germplasm is characterized by the presence of sequences representing the sequences at polymorphic sites (e.g., SNPs) that are homozygous or heterozygous. As used herein, the germplasm of the present disclosure is characterized by the homozygous loci. In haploid cells (e.g., ova or sperm), each of the sequences at each homozygous locus in the parent are present in each cell. In contrast, heterozygous loci are present in haploid germplasm according to random assortment. Accordingly, the genotype at heterozygous loci in haploid cells varies from haploid cell to haploid cell, while the sequences of the homozygous loci from the parent are present in each haploid cell and are therefore faithfully transmitted to progeny.

During breeding, to maintain and improve the germplasm, selections of a second parent may be an elite second parent having superior traits. As demonstrated in the examples, such select crosses result in progeny that retain significant numbers of homozygous loci present in each elite parent and further result in additional loci becoming homozygous. Thus, select crosses result in some or all of the homozygous loci of the Bos taurus cells of the present disclosure being retained in progeny generations. Generally, locus homozygosity is maintained when the second parent is an elite parent with high trait values as described below. Similarly, the improved germplasm of the present disclosure can be maintained by selecting superior breeding partners in the F1 and later progeny generations. By suitable selection of the second parent, the germplasm can be maintained at the homozygous loci. Even further, careful selection of elite breeding partners results in increased numbers of homozygous loci as the preferred allele at the heterozygous loci of the parent become homozygous in elite progeny.

As used herein, “gamete” refers to a haploid germ cell and includes either a sperm or an ovum and may be used interchangeably. Generally, the identity as a sperm or an ovum can be determined by the context as bulls produce sperm and cows produce ova. For the purposes of this disclosure the term “sperm” means the haploid cell that is the gamete of a male animal which may join an egg (also referred to as “ovum”) to produce a zygote and broadly encompasses infertile sperm, sperm having a comparably lesser or a comparably greater fertility between a first amount of sperm obtained from a first animal and a second amount of sperm obtained from a second animal and which may be obtained in the form of a raw ejaculated semen, frozen semen, as sperm separated from the semen and contained in an extender or diluent, or as sex-selected sperm.

As used herein, the term “inseminate” is intended to broadly encompass an amount of sperm whether contained in semen alone or together with a cryoprotectant, extender, or other diluent. Inseminates may optionally include one or more “extenders” and diluents which can be utilized to fertilize the eggs of a female animal whether in vitro or in vivo. As used herein, inseminates can further include sex-selected sperm compositions.

As used herein, the term “sex-selected sperm” means sperm which have been separated, regardless as to the method of separation, into subpopulations containing X-chromosome bearing sperm and Y-chromosome bearing sperm having a purity in the range of about 70 percent (“%”), 80%, 90%, and about 100%.

As used herein, the term F1 refers to the first filial generation—progeny born from the gametes of a particular parent. Therefore, as used herein, an F1 Animal is a direct genetic reproduction descendent of Animal. Because cattle reproduce by meiosis, this term refers to an animal arising from a gamete of the parent—in this case, from a gamete from Animal. Therefore, for each of Animal's chromosome pairs, their offspring, an F1 Animal, will receive one copy of the chromosome carrying one of Animal's alleles for each locus. In general, this means that for each locus, an F1 Animal will receive one allele from Animal and one from their other parent. An F1 Animal, therefore, will receive one copy of each of Animal's homozygous alleles, and, for each locus, one copy of one of Animal's heterozygous alleles. This means that two progeny of Animal with the same second parent will not necessarily inherit the same heterozygous alleles from Animal and may have varying genotypes for Animal's heterozygous loci.

### Animal Genotype Characterizations

The present disclosure provides for, and includes, cells, animals, and progeny of Bos taurus Animal comprising an improved germplasm characterized by the Animal Genotype and homozygous loci comprising the nucleic acid sequences of Animal Homozygous Genotype. Animal differs from the reference genome at 11645 homozygous loci, or about 28% of the total loci. All chromosome positions are determined using the ARS-UCD1.2 October, 2019 bovine genome build.

TABLE 1
Genotype of Bos taurus Animal

| | SEQ ID Range | | |
|---|---|---|---|
| | SEQ ID NOs: 1 to 28548 | | No. of |
| | Reference | | Alternate |
| Homozygous | Allele | Alternate Allele | Alleles |
| Chromosome 1 | 1-1015 | 16875-17556 | 681 |
| Chr. 2 | 1016-1888 | 17557-18185 | 628 |
| Chr. 3 | 1889-2772 | 18186-18690 | 504 |
| Chr. 4 | 2773-3563 | 18691-19237 | 546 |
| Chr. 5 | 3564-4256 | 19238-19681 | 443 |
| Chr. 6 | 4257-4995 | 19682-20318 | 636 |
| Chr. 7 | 4996-5785 | 20319-20884 | 565 |
| Chr. 8 | 5786-6581 | 20885-21432 | 547 |
| Chr. 9 | 6582-7201 | 21433-21909 | 476 |
| Chr. 10 | 7202-7906 | 21910-22452 | 542 |
| Chr. 11 | 7907-8579 | 22453-22981 | 528 |
| Chr. 12 | 8580-9102 | 22982-23353 | 371 |
| Chr. 13 | 9103-9702 | 23354-23712 | 358 |
| Chr. 14 | 9703-10234 | 23713-24129 | 416 |
| Chr. 15 | 10235-10807 | 24130-24499 | 369 |
| Chr. 16 | 10808-11353 | 24500-24885 | 385 |
| Chr. 17 | 11354-11847 | 24886-25270 | 384 |
| Chr. 18 | 11848-12351 | 25271-25552 | 281 |
| Chr. 19 | 12352-12774 | 25553-25872 | 319 |
| Chr. 20 | 12775-13336 | 25873-26288 | 415 |
| Chr. 21 | 13337-13808 | 26289-26590 | 301 |
| Chr. 22 | 13809-14285 | 26591-26904 | 313 |
| Chr. 23 | 14286-14627 | 26905-27134 | 229 |
| Chr. 24 | 14628-15078 | 27135-27459 | 324 |
| Chr. 25 | 15079-15423 | 27460-27632 | 172 |
| Chr. 26 | 15424-15826 | 27633-27854 | 221 |
| Chr. 27 | 15827-16183 | 27855-28133 | 278 |
| Chr. 28 | 16184-16499 | 28134-28355 | 221 |
| Chr. 29 | 16500-16874 | 28356-28548 | 192 |
| | | No. Alternate | 11645 |
| | | alleles | |

| | SEQ ID |
|---|---|
| Heterozygous | Range |
| Chromosome 1 | 28549-29513 |
| Chr. 2 | 29514-30221 |
| Chr. 3 | 30222-30874 |
| Chr. 4 | 30875-31534 |
| Chr. 5 | 31535-32165 |
| Chr. 6 | 32166-32832 |
| Chr. 7 | 32833-33299 |
| Chr. 8 | 33300-33865 |
| Chr. 9 | 33866-34415 |
| Chr. 10 | 34416-34914 |
| Chr. 11 | 34915-35524 |
| Chr. 12 | 35525-36005 |
| Chr. 13 | 36006-36476 |
| Chr. 14 | 36477-36973 |
| Chr. 15 | 36974-37414 |
| Chr. 16 | 37415-37794 |
| Chr. 17 | 37795-38218 |
| Chr. 18 | 38219-38522 |
| Chr. 19 | 38523-38891 |
| Chr. 20 | 38892-39178 |
| Chr. 21 | 39179-39497 |
| Chr. 22 | 39498-39740 |
| Chr. 23 | 39741-40051 |
| Chr. 24 | 40052-40299 |
| Chr. 25 | 40300-40592 |
| Chr. 26 | 40593-40853 |
| Chr. 27 | 40854-41023 |
| Chr. 28 | 41024-41255 |
| Chr. 29 | 41256-41546 |

The present disclosure provides for, and includes, a diploid Bos taurus cell or a plurality of diploid Bos taurus cells comprising improved germplasm characterized by a genome having homozygous loci comprising 90% to 100% of the nucleic acid sequences characterizing the Animal Homozygous Phenotype.

### Phenotypic Traits and Indices

Predicted Transmitting Abilities (PTAs) can be computed for various traits, for example in the broad categories of production (milk and milk components), health/fitness, and type. Dairy cattle are evaluated for the traits of milk, fat, and protein yield, length of productive life, and somatic cell score (an indicator of mastitis). Evaluation procedures combine information from relatives of an evaluated animal, and from the animal itself in the case of cows. Additionally, numerous type or conformation traits are evaluated routinely.

An important aspect of Bos taurus breeding programs are genetic values for yield, management traits, and type that are reported as PTAs. These important traits include higher milk, protein and fat yield PTAs shown as added pounds of milk, fat, and protein expected per lactation for average daughters of individual sires. PTAs are also expressed as percentage traits that are represented as percentage point differences where plus values indicate higher concentration of fat and protein in milk. Bos taurus animals and cells obtained therefrom are also selected to provide increased numbers of daughters and herds contributing to production, improved reliability, improved productive life, improved somatic cell score (SCS). Desirable Bos taurus varieties provide higher daughter pregnancy rates (DPR) that are a genetic measure of the percentage of non-pregnant cows that become pregnant during each 21-day period (heat cycle). Another important and desirable PTA selected by breeders is the productive life (PL) that is the additional months of life in the milking string. Also included among the desirable PTAs are calving related traits such as the service sire calving ease and the service sire stillbirth.

Traits are typically combined into an index based on their relative economic weights. For example, the Net Merit index (NM$) computed by USDA AIPL in conjunction with CDCB estimates lifetime profit based on incomes and expenses relevant for today's dairy producers and is expressed as a dollar value. Traits included in NM$ are: protein (lb.), fat (lb.), productive life, somatic cell score, udder composite, feet/legs composite, body size composite and daughter pregnancy rate. Calving ability also is included in NM$ calculations for Holsteins and Brown Swiss. The traits incorporated into calving ability for Holsteins are daughter stillbirth, service sire stillbirth, daughter calving ease, and service sire calving ease. Only the two calving ease traits are available for inclusion in calving ability values of Brown Swiss. Net merit scores are a relative score calculated based on a comparison with a baseline average animal in 2015. A new baseline animal based on the average animal in 2020 is expected to begin use in 2025 and will reflect improvements to dairy herds generally. For example, NM$ scores in 2020 were reduced by 231 dollars compared to earlier NM$ scores. As used herein, NM$ scores are calculated using the 2015 baseline and persons of ordinary skill in the art will be to calculate or convert NM$ values based on the 2015 baseline population. Methods to calculate NM$ are known in the art. See VanRaden et al., “Net merit as a measure of lifetime profit: 2018 revision,” USDA AIP Research Report NM$7 (5-18) (2018) available at aipl(dot)arsusda(dot)gov/reference/nmcalc-2018.htm for exemplary purposes only (this recent publication uses updated baseline animals not necessarily in use by the industry for Net Merit calculations.) The NM$ incorporates production, management and important composite traits and is designed for dairies paid for protein plus fat and requiring more emphasis on management traits. The present disclosure provides for, and includes, increased NM$ of progeny cattle relative to parents. In an aspect, the NM$ of progeny cattle is increased by at least 10% over the parent generation. Also included are methods to improve NM$ in a herd comprising crossing an Animal of the present disclosure with individuals of a herd in need of improvement.

Trait parameters have been correlated with the underlying genetics and the heritabilities determined. The genetic and phenotypic correlations among the twelve PTA traits are also provided by VanRaden et al., 2018.

Holstein Association USA (HAU) calculates the Total Performance Index (TPI®; HAU, Brattleboro, Vt.)). It includes the traits of protein, fat, type, udder composite, feet and leg composite, daughter pregnancy rate, productive life, somatic cell score, daughter calving ease, daughter stillbirth and dairy form. Like Net Merit scores, TPI® is calculated based on a comparison with a baseline average animal in 2010, however, the Holstein Association USA has not announced plans to update the baseline animal. Accordingly, as used herein, TPI® scores are calculated using a 2010 baseline animal. However, should a new baseline year be adopted, persons of ordinary skill in the art will be able to calculate or convert TPI® values based on the 2010 baseline population for any future baseline animal using methods known in the art (Available on the internet at www(dot)holsteinusa(dot)com/genetic_evaluations/ss_tpi_formula.html.) TPI® (HAU, Brattleboro, Vt.) incorporates production, management, Type, and important linear and composite traits. TPI® focuses on dairies paid for protein plus fat and requiring more emphasis on Type.

Using a selection index can be an effective way to consider several traits when choosing breeding stock. Conventional animal husbandry strategies often rely on selection indexes, particularly for choosing service sires.

Breeding stock sires and sire lines are typically chosen based upon their size and fertility. Prior successes in mating as well as siring females are both traits that are often utilized in selecting sires and/or sire lines.

Also, knowing where service sires rank relative to other active bulls is typically considered to be helpful in determining if the sires meet a particular herd's genetic goals. Selection indexes can be particularly useful in monitoring such ranking. To maximize genetic improvement using a selection index, it is usually recommended that the service sires for a given herd average at or above the 80th percentile.

In order to provide the most up to date information, the Council of Dairy Cattle Breeding publishes updated sire information 3 times a year, in April, August, and December. Skilled persons refer to these publications as “Sire Summaries.”

In germplasm improvement programs, “Dams of Males” (DM) or more commonly, “bull mothers” represent a group of elite females that are selected based on estimated breeding values (EBV) or genomic breeding values (GEBV) and that usually rank among the top 1% of the population. These elite females are typically mated to elite bulls from the “Sires of Males” (SM) group for the purpose of producing elite bull calves.

“Sires of males” (SM) or “sires of sons” are elite males that are also selected by EBV or GEBV to be sires of the next generation of young elite bulls and represent <5% of the males. It is the SM semen that is marketed to dairy farmers and it is primarily through the distribution of SM semen that commercial herds are improved.

Sires of females (SF) or “active AI sires” represent a larger group of males that have been selected based on EBV or GEBV and whose semen is used to breed the general population and produce replacement females for commercial farms.

“Dams of Females” (DF) or “commercial cows” represent the large population of females that are primarily used to produce milk rather than breeding stock and are routinely mated to bulls from the “Sires of Females” (SF) group to initiate lactation, resulting in the next generation of replacement heifers.

Germplasm development programs are directed to the continued improvement of the elite (DM and SM) individuals that in turn improve the germplasm of the larger DF and SF populations. Distinct DM and SM lines have been developed to serve different markets and the resulting germplasms are distinct. That is, different DM and SM lines have distinctive sets of alleles as a result of multigenerational selections.

Ranchers and dairy farmers increase the value of a cow's calves by utilizing frozen semen from the most valuable SM bulls in the industry to breed their cows. Frozen semen can be shipped commercially around the world, the best bulls can be mated to thousands of cows instead of the usual 20 to 40 under natural pasture mating conditions. Once thawed, the semen can be used as an inseminate.

As used herein, daughter pregnancy rate (DPR) is a genetic measure of the percentage of non-pregnant cows or heifers eligible to become pregnant that actually become pregnant during each 21-day period (heat cycle). DPR is similar to, but not always the same as, pregnancy rates computed for herd management purposes. Daughters of sires which have larger PTA DPR are more likely to conceive during a given heat cycle and each 1% increase in PTA DPR is associated with a genetic decrease of 4 days open.

As used herein, Cow Livability (LIV) represents the probability value of a lactation not ending in death or euthanasia relative to the average of the breed. This trait is important because cows that die during lactation have no value and the farmer must pay to dispose of the carcass. The trait is similar to PL which includes cows culled from the herd for any reason. LIV values range from about −5 to +5, where 5% more of a bull's daughters will remain alive compared to the breed average.

As used herein, heifer conception rate (HCR) is the percentage of inseminated heifers that become pregnant at each service, shown as a deviation in percentage.

As used herein, cow conception rate (CCR) is the percentage of inseminated cows that become pregnant at each service, shown as a deviation in percentage.

As used herein, estimated future inbreeding (EFI) is the estimate of future progeny inbreeding, assuming that an animal is mated randomly within their given breed.

As used herein, kappa casein (K-casein) identify sires with homozygous BB genotype for Kappa Casein for preferred cheese production.

As used herein, predicted transmitting ability protein (PTAP) is a yield trait for protein measured in pounds that is the predicted difference of the protein yield of the offspring from the average. PTAP is shown as added pound of protein expected per lactation for average daughters of individual sires. Higher numbers are preferred.

As used herein, predicted transmitting ability fat (PTAF) is a yield trait for fat measured in pounds that is the predicted difference of the fat yield of the offspring from the average. PTAF is shown as added pound of fat expected per lactation for average daughters of individual sires. Higher numbers are preferred.

As used herein, predicted transmitting ability milk (MILK or PTA MILK) is a yield trait for milk measured in pounds that is the predicted difference of the milk yield of the offspring from the average. MILK is shown as added pounds of milk expected per lactation for average daughters of individual sires. Higher numbers are preferred.

As used herein, feed efficiency value (FE) recognizes cattle with the ability to produce large volumes of milk without having to consume a great deal of feed, based on the following formula: Feed Efficiency=(Dollar value of milk produced)−(Feed costs for extra milk)−(Extra maintenance costs).

As used herein, predicted transmitting ability type (PTAT or TYPE) is represented as differences in points from a base population physical conformation. Daughter final scores are collected by breed classifiers. Raw scores then are adjusted for cow age and used to derive Type PTAs (PTAT). These PTAT are represented as differences in points from the base population. TYPE values are normalized to enable comparisons across different base populations through time. Higher numbers correlate with more desirable physical conformations.

As used herein, STA dairy form (DF or DFM), formerly known as “dairy character”, refers to sharpness, angularity, flatness of bone, openness of rib and length of neck that provides an indication of ‘milkiness’ and reflects the ability of a dairy cow to produce milk from the feed over flesh and fat.

Dairy cattle are evaluated and described using criteria generally known as linear descriptive traits that are well known in the art. These linear descriptive traits include Stature (STA), Strength (STR), Body Depth (BDE), Rump Angle (RPA), Thurl Position, Rump Width, Fore Udder Height (FTA), Fore Udder Attachment (FUA), Rear Udder Height (RUH), Rear Udder Width (RUW), Udder Cleft (UCL), Udder Depth (UDP), Front Teat Placement (FTP), Rear Teat Placement (RTP), Teat Length (TLG), Udder Tilt (UT), Rear Legs (Side View) (RLS), Rear Legs (Rear View) (RLR), Feet Leg Score (FLS), Foot Angle, Thurl Width (TRW), and Body Condition. These linear trait criteria are well known to those skilled in the art. See for example, The Dairy Cow Today: U.S. Trends, Breeding, and Progress Since 1980, S. L. Spahr and G. W. Opperman, Chapter 9, Type and classification and trait appraisal, hereby incorporated by reference in its entirety.

Linear descriptive traits are often combined into composite indexes to simplify the process of describing the transmitting pattern for type traits. Composite indexes include the feet & legs composite (FLC), the udder composite index (UDC), the body form (BF) composite index, body size composite (BSC) index, and the dairy capacity (DC) composite index. The FLC composite is a combination of rear legs, side view and foot angle linear traits. The UDC incorporates the udder attachment, rear udder height, rear udder width, udder depth, udder cleft, front teat placement, and rear teat placement linear traits. The UDC is designed so that the association between UDC and herd life is maximized. Larger values are associated with longer herd life. The udder composite index describes a well formed capacious udder with strong attachment. Using breeding animals with a high UDC results in a lowering of the somatic cell score and daughters whose udders are trouble-free and capable of holding more milk. The BF index combines the linear traits of stature, body depth, rump angle, and rump width. The DC composite combines the linear traits of dairy form and strength. The BSC is another composite index calculated from four linear traits: stature, strength, body depth and rump width. Every 1.0 STA increase in the BSC correlates with a 24 pound predicted increase in mature body weight.

As used herein, “Productive Life” (PL) is a measure of how long dairy cows survive in a herd after they calve for the first time. It is based on calving dates, culling or death dates, and days in milk (based on dry dates) in each lactation for cows on DHI test. The PTA for Productive Life (PL) is expressed as additional months of life in the milking string. Bulls with larger PL are expected to sire daughters that have longer productive lives. Data used to compute PL include actual longevity, stage of lactation, and culling data supplemented with data from traits that are correlated with PL. By assigning the largest PL credits for months in peak production and by giving later lactations slightly more credit than first lactation, PL reflects the economic impact of cow longevity. The heritability of PL is low at 0.085 and the trait is expressed late in the life of dairy cow. Accordingly, PL is a difficult trait to improve through selection because of low heritability and expression of the trait late in life. Methods for calculating PL are known in the art. See VanRaden et al., Journal of Dairy Science 76:2758-2764 (1993), VanRaden et al., Journal of Dairy Science 78:631-638 (1995), Weigel et al., Journal of Dairy Science 81:2040-2044 (1998). Each of the foregoing references are hereby incorporated by reference in their entireties. See also VanRaden et al., “Methods used to compute multi-trait productive life,” USDA AIPL Research Report PLC (11-03) available at aipl(dot)arsusda(dot)gov/reference/multi-pl.htm. The present disclosure provides for, and includes, progeny cattle having an increased PL relative to a dam parent.

As used herein, “Somatic Cell Score” (SCS) is calculated from the Somatic Cell Count (SCC). When milk is produced, a small number of cells are also transferred to the milk (along with the proteins, fat, water, and minerals that make up milk.) Although all milk contains some of these cells, milk quality is affected if they are present in very high numbers. Milk processors limit the concentration of cells that they will allow in milk they buy from farmers. Also, knowing the SCS for an individual cow can help the farmer tell if the cow is healthy because irritation in the udder can cause higher SCS. Health management has the biggest effect on SCS, but just like some people inherit a higher chance of getting ear infections, cows can inherit traits which cause higher SCS. Next to traits like milk or protein production, SCS has a low heritability. Somatic Cell Score PTA is calculated using somatic cell score data from the first five lactations as an indicator of mastitis resistance. Bulls with the lowest PTA SCS are expected to sire daughters with the lowest SCS, the lowest somatic cell counts (SCC), and the fewest cases of mastitis. The present disclosure provides for, and includes, reduced SCS in progeny compared to a dam parent.

As used herein, “Fertility Index” (FI) combines several reproductive components into one overall index: ability to conceive as a heifer, ability to conceive as a lactating cow, and a cow's overall ability to start cycling again, show heat, conceive, and maintain a pregnancy. The Fertility Index is derived from the formula: Fertility Index=18% Heifer Conception Rate (HCR)+18% Cow Conception Rate (CCR)+64% Daughter Pregnancy Rate (DPR). The present disclosure provides for, and includes, an increased FI in progeny compared to a dam parent. Also included are methods to improve FI in an offspring comprising crossing an Animal of the present disclosure with dams of a herd in need of improvement.

As used herein, “Sire Calving Ease” (SCE) measures the tendency of calves from a particular sire to be born more or less easily and is expressed as a percent of difficult births in first calf heifers on a scale of 1 to 5 (1 is classified as “no problem”). The percent difficult birth among Holstein is about 8%. Generally, bulls having an SCE of 8% or less are considered “calving ease” bulls. Lower numbers are preferred. The present disclosure provides for, and includes, reduced SCE of progeny cattle. Also included are methods to improve SCE in a progeny comprising crossing an Animal of the present disclosure with individuals of a herd in need of improvement.

As used herein, “Daughter Calving Ease” (DCE), like SCE, is a measurement of the tendency of calve from a particular animal to be born more or less easily. Lower numbers are preferred. The present disclosure provides for, and includes, reduced DCE of progeny cattle. Also included are methods to improve DCE in a progeny comprising crossing an Animal of the present disclosure with individuals of a herd in need of improvement.

As used herein, “Service Sire Stillbirth” (SSB) expresses the proportion of stillborn calves expected from sires. The genetic base for Stillbirth is 8%. The present disclosure provides for, and includes, reduced SSB of progeny cattle relative to the genetic base. In an aspect, the progeny cattle have an SSB below 5%. Also included are methods to improve SSB in a progeny comprising crossing an Animal of the present disclosure with individuals of a herd in need of improvement.

As used herein, “Daughter Stillbirth” (DSB) is the tendency of calves from a sire to be stillborn and applies to the Holstein breed only. As discussed below, DSB can be related to certain haplotypes. The present disclosure provides for, and includes, reduced DSB of progeny cattle relative to the genetic base. In an aspect, the progeny cattle have a DSB below 5%. Also included are methods to improve DSB in a progeny comprising crossing an Animal of the present disclosure with individuals of a herd in need of improvement.

Breeders have developed merit measures for the evaluation of value of improved Bos taurus germplasm over the lifetime of offspring. Various merit measures account for the additional net profit that an offspring of an animal will provide over its lifetime. Income and expenses for a typical dairy operation have been estimated, so that a measure of overall net profit can be calculated. Three different values (Net, Fluid and Cheese) of lifetime profitability are available. The primary difference between the formulas is the emphasis that is placed on the components. When breeding, producers select the index that is closest to the milk payment in their area. Net merit is based upon the future anticipated average milk price for all of the United States. Fluid Merit would be for producers who do not receive any payment for protein. In the Fluid Merit formula, a negative value is placed on protein because additional feed is required to produce additional protein. Without a direct payment for the additional protein, this results in a negative value. Cheese Merit may be appropriate for farmers selling their milk directly to a cheese plant.

As used herein, grazing merit (GM$) is an index that incorporates economic values appropriate for grazing production in the U.S. The GM$ index is based upon appropriate costs and revenues to allow for selection of cows and bulls for more optimal genetic progress. GM$ is geared toward herds on pasture systems, with those breeders often demanding higher fertility, compared to conventional systems, due to seasonal calving requirements. Methods to calculate GM$ are known in the art. See Gay et al., J. Dairy Sci. 97:4568-4578 (2014), hereby incorporated by reference in its entirety. The present disclosure provides for, and includes, increased GM$ of progeny cattle relative to parents. In an aspect, the GM$ of progeny cattle is increased by at least 10% over the parent generation. Also included are methods to improve GM$ in a herd comprising crossing an Animal of the present disclosure with individuals of a herd in need of improvement.

As used herein cheese merit (CM$) is an index that incorporates economic values appropriate for milk sold to be made into cheese or other dairy products. The formula incorporates MILK, PTAF, PTAP, and various health and type traits. A discussion of CM$ is available on us(dot)altagenetics(dot)com. The present disclosure provides for, and includes, increased CM$ of progeny cattle relative to parents. In an aspect, the CM$ of progeny cattle is increased by at least 10% over the parent generation. Also included are methods to improve CM$ in a herd comprising crossing an Animal of the present disclosure with dams of a herd in need of improvement.

As used herein, fluid merit (FM$) is an index that incorporates economic values for dairy production wherein the producers do not receive any payment for protein. Methods for calculating FM$ are known in the art. The present disclosure provides for, and includes, increased FM$ of progeny cattle relative to parents. In an aspect, the FM$ of progeny cattle is increased by at least 10% over the parent generation. Also included are methods to improve FM$ in a herd comprising crossing an Animal of the present disclosure with individuals of a herd in need of improvement.

As used herein, a haplotype is a combination of alleles (DNA sequences) at different locations on a chromosome that are transmitted together as a group (linked). Haplotype tests are available that provide for the identification of recessive disorders that affect fertility and other traits. See Cole et al., “Haplotype tests for recessive disorders that affect fertility and other traits,” USDA AIP Research Report Genomic3 (09-13) updated Dec. 1, 2018, available at aipl(dot)arsusda(dot)gov/reference/recessive_haplotypes_ARR-G3.html. Certain haplotypes are undesirable in a Bos taurus germplasm as provided in the present specification. When the recessive haplotype is homozygous, fertility and other critical traits are significantly affected. The germplasm of the present disclosure can be used to improve herds and reduce the presence of these undesirable haplotypes.

Undesirable recessive haplotype mutations include polledness (lack of horns—haplotype HHP in Holsteins, RIP in Jerseys which is currently associated with other adverse phenotypes, but may become desirable as it removes the need to dehorn animals), and red coat color (Holsteins are supposed be black and white—haplotypes HBR, HDR, and HHR). Several haplotypes do not produce viable homozygous offspring: HH0, HH1, HH2, HH3, HH4, and HH5 in Holstein cows. HH0 causes Brachyspina syndrome (HH0), which is a congenital inherited lethal defect in Holstein cattle that causes embryonic death, stillbirth and other deformities. (e.g., TY). HH1 is a nonsense mutation in the APAF1 gene. HH3 is caused by an SNP in the SMC2 gene causing a single amino acid substitution. HH4 is caused by genetic lesions in the GART gene. HH5 is caused by genetic lesions in the TFB1M gene. JH1 is a mutation in CWC15. The genetic defect associated with HH2 is still unknown.

Cattle suffer from a number of genetic diseases that are monogenic disorders inherited in a Mendelian fashion. Various genetic diseases are known in the art. See for example, Garrick and Ruvinsky, “The Genetics of Cattle,” 2nd Edition, CAB International, Oxfordshire UK 2015; see also Parkinson et al., “Diseases of Cattle in Australasia,” ISBN 9780958363447 Jolly et al., “Genetic Diseases of Cattle,” Chapter 21, each hereby incorporated by reference in their entireties, and vetbook(dot) org/wiki/cow/index.php/Genetic_diseases_of_cows. The germplasm of the present disclosure can be used to reduce the presence of these recessive genes in herds and bovine populations.

Several other health traits are tracked in Holstein cattle, including hypocalcemia (milk fever), Displaced Abomasum (DA), ketosis, mastitis, metritis, and retained placenta. Genomic and genetic evaluations are provided for Holstein animals; the evaluation of each trait is expressed in percentage points of resistance above or below the breed average.

Animal Genotype provides for, and includes, a diploid Bos taurus cell or a plurality of diploid Bos taurus cells comprising improved germplasm characterized by a genome having 90% to 100% of homozygous loci characterizing the Animal Homozygous Genotype. The present disclosure provides for, and includes, a diploid Bos taurus cell or a plurality of diploid Bos taurus cells comprising improved germplasm characterized by a genome having 90% to 100% of homozygous loci characterizing the Animal Homozygous Genotype. In an aspect, the diploid Bos taurus cell or a plurality of diploid Bos taurus cells comprise a genome having 90% to 100% of homozygous loci characterizing the Animal Homozygous Genotype. In an aspect, the genome of said Bos taurus cell or cells further comprise at least 90% of heterozygous loci characterizing the Animal Heterozygous Genotype. In an aspect, the plurality of cells comprises a frozen vial, a cell culture, a tissue, a zygote, an embryo, a calf, or a mature adult. In some aspects, the Bos taurus cells are non-reproductive cells. In various aspects, the Bos taurus cells are gametes. In an aspect, the number of cells in a plurality of cells of the present disclosure is two or more, 100 or more, 1000 or more, 10⁴ or more, 10⁵ or more, or 10⁶ or more. In an aspect, the cells are in a container and comprise between 10⁴ and 10⁷ cells. In an aspect, the number of cells is between 10⁵ and 10⁷ cells.

In other aspects, the present disclosure provides for and includes, a diploid Bos taurus cell or a plurality of diploid Bos taurus cells comprising improved germplasm characterized by a genome comprising at least 95% of homozygous loci characterizing the Animal Homozygous Genotype. In an aspect, the diploid Bos taurus cell or plurality of diploid Bos taurus cells further comprise at least 95% of heterozygous loci characterizing the Animal Heterozygous Genotype. In another aspect, the diploid Bos taurus cell or plurality of diploid Bos taurus cells comprise a genome comprising 97% of homozygous loci characterizing the Animal Homozygous Genotype. In an aspect, the diploid Bos taurus cell or plurality of diploid Bos taurus cells further comprise 97% of heterozygous loci characterizing the Animal Heterozygous Genotype. In another aspect, the diploid Bos taurus cell or plurality of diploid Bos taurus cells comprise a genome comprising at least 98% of homozygous loci characterizing the Animal Homozygous Genotype. In an aspect, the diploid Bos taurus cell or plurality of diploid Bos taurus cells further comprise at least 98% of heterozygous loci characterizing Animal Heterozygous Genotype. In another aspect, the diploid Bos taurus cell or plurality of diploid Bos taurus cells comprise a genome comprising at least 99% of homozygous loci characterizing the Animal Homozygous Genotype. In an aspect, the diploid Bos taurus cell or plurality of diploid Bos taurus cells comprise at least 99% of heterozygous loci characterizing the Animal Heterozygous Genotype. In another aspect, the diploid Bos taurus cell or plurality of diploid Bos taurus cells comprise a genome comprising at least 99.5% of homozygous loci characterizing the Animal Homozygous Genotype. In an aspect, the diploid Bos taurus cell or plurality of diploid Bos taurus cells further comprise at least 99.5% of heterozygous loci characterizing the Animal Heterozygous Genotype. In an aspect, the diploid Bos taurus cell or plurality of diploid Bos taurus cells comprise a genome comprising homozygous loci characterizing the Animal Homozygous Genotype. In an aspect, the diploid Bos taurus cell or plurality of diploid Bos taurus cells further comprise heterozygous loci characterizing the Animal Heterozygous Genotype. In an aspect, the diploid Bos taurus cells comprise Bos taurus Animal, wherein a sample of cells of Animal comprises the Deposit. As provided herein, the plurality of cells may comprise a frozen vial, a cell culture, a tissue, a zygote, an embryo, a calf, or a mature adult. In some aspects, the Bos taurus cells are non-reproductive cells. In an aspect, the number of cells in a plurality of cells of the present disclosure is two or more, 100 or more, 1000 or more, 10⁴ or more, 10⁵ or more, or 10⁶ or more. In an aspect, the cells are in a container and comprise between 10⁴ and 10⁷ cells. In an aspect, the number of cells is between 10⁵ and 10⁷ cells.

The present disclosure provides for, and includes, tissue cultures comprising diploid Bos taurus cells comprising improved germplasm characterized by a genome comprising an Animal Genotype. In an aspect, the culture of Bos taurus cells comprises a genome comprising homozygous loci comprising the nucleic acid sequences of the Animal Homozygous Genotype. As provided above, tissue cultures may comprise cells having 95% of the recited homozygous loci, 97% of the recited homozygous loci, 98% of the recited homozygous loci, 99% of the recited homozygous loci, 99.5% of the recited homozygous loci, or 100% of the recited homozygous loci. In an aspect, the diploid Bos taurus cell culture comprises Bos taurus Animal, wherein a sample of cells of Animal comprises the Deposit. As provided herein, the cell culture may be prepared from frozen cells or obtained as a primary culture from an embryo, a calf, or a mature adult. In certain aspects, the Bos taurus cells are non-reproductive cells.

Also included, and provided for, are Bos taurus cells that are haploid. Animal Haploid Genotype provides for, and includes, a haploid Bos taurus cell or a plurality of haploid Bos taurus cells comprising improved germplasm characterized by a genome having 90% to 100% of loci characterizing the Animal Homozygous Genotype. As described below, haploid cells can be readily produced by somatic cell nuclear transfer of Bos taurus Animal, wherein a sample of cells of Animal comprises the Deposit. Mature animals prepared from Animal can be used to obtain haploid gametes through methods known in the art and discussed in detail below. As used herein, the term “gametes,” “sperm,” “spermatozoa,” “spermatid,” and “inseminate” are used interchangeably for haploid cells obtained from male animals and the terms will be understood by a person of ordinary skill in the art. As used herein, the term “gametes,” “ova,” and “ovum” are used interchangeably for haploid cells obtained from female animals. Haploid Bos taurus cells of the present disclosure comprise a haploid genome comprising a single copy of at least 90% of loci characterizing the Animal Homozygous Genotype. Haploid cells of the present disclosure include a haploid genome comprising a single copy of at least 95% of loci characterizing the Animal Homozygous Genotype. Haploid cells of the present disclosure also include a haploid genome comprising a single copy of at least 97% of loci characterizing the Animal Homozygous Genotype. Haploid cells of the present disclosure may also comprise a haploid genome comprising a single copy of at least 98% of loci characterizing the Animal Homozygous Genotype. Haploid cells of the present disclosure include a haploid genome comprising a single copy of at least 99% of loci characterizing the Animal Homozygous Genotype. In some aspects, haploid cells of the present disclosure comprise a haploid genome comprising a single copy of at least 99.5% of loci characterizing the Animal Homozygous Genotype. Haploid cells of the present disclosure also include cells comprising a haploid genome comprising a single copy of loci characterizing the Animal Homozygous Genotype. In an aspect, the number of haploid cells from male animals in a plurality of cells of the present disclosure is two or more, 100 or more, 1000 or more, 10⁴ or more, 10⁵ or more, or 10⁶ or more. In an aspect, the cells are in a container and comprise between 10⁴ and 10⁷ cells. In an aspect, the number of cells in a container is between 10⁵ and 10⁷ cells.

As provided herein, the plurality of haploid cells may be cells in fresh semen. Methods for the collection of sperm from mature bulls are well known in the art.

Bos taurus germplasms are often provided as frozen (cryopreserved) semen samples. A long-term semen storage system is advantageous by allowing movement of artificial insemination (AI) doses throughout the world without compromising semen viability. Long-term storage also enables specific health checks to be carried out on both the semen and individual males thus minimizing the risk of the spread of disease through AI. Cryopreservation techniques for semen are well known to those skilled in the art.

As provided herein, useful cryoprotectants are not limited to those acting by a particular mechanism. In an aspect, the cryoprotectant acts, at least in part, by reducing intracellular dehydration. Not to be limited by theory, freezing is accompanied by an increase in solute concentration in the medium surrounding sperm that draws water out of the cells leading to increases in intracellular electrolyte concentration. Cryoprotectants of the present disclosure include, but are not limited to glycerol (GLY), 1,1,1-tris(hydroxymethyl)ethane [2-hydroxymethyl-2-methyl-propane-1,3-diol] (THE), 1,1,1-tris(hydroxymethyl)propane [2-ethyl-2-hydroxymethyl-propane-1,3-diol] (THP), ethylene glycol (EG), propane-1,2-diol (PD2), propane-1,3-diol (PD3) and dimethylsulphoxide (DMSO), sucrose, trehalose, dextrose, raffinose, lactose, melibiose, melezitose, mannotriose, stachyose, dextran, hydroxy-ethyl starch, maltitol, lactitol, polyethyleneglycol, propylene glycol, polyvinvyl pyrrolidone, polyethylene oxide, and combinations thereof.

The present disclosure provides for, and includes, containers of Bos taurus inseminates comprising a plurality of sperm cells. Inseminates are prepared by combining fresh semen samples with a cryoprotectant and freezing the samples. Upon thawing, the sperm retain high levels of motility and fertility. In addition to the cryoprotectant, inseminates often include one or more additional components. In an aspect, the inseminate comprises 10⁴ or more, 10⁵ or more, or 10⁶ or more cells. In an aspect, the inseminate is provided in a container and comprises between 10⁴ and 10⁷ cells. In an aspect, the number of cells in the container is between 10⁵ and 10⁷ cells.

In general, about 5 ml to about 15 ml of semen is collected from a bull and optionally mixed with a suitable extender and cryoprotectant. For example, in an aspect, about 10 ml of semen is collected and mixed with about 240 ml of TRILADYL™ (Minitube, Verona, Wis.) solution, which is an off the shelf product that is available from Minitube of America in Verona, Wis. The TRILADYL™ contains an extender and a cryoprotectant, such as glycerol. The mixture of semen, extender and cryoprotectant is then placed in plastic straws and frozen. In the industry the contents of the frozen straw are generally referred to as frozen semen, although the contents also contain an extender and a cryoprotectant. A goal is to cryopreserve about 20 million motile sperm in a ½ ml semen straw.

As used herein, an inseminate refers to a composition of Bos taurus sperm having an Animal Haploid Genotype and a cryoprotectant. Also provided are inseminates further comprising a component selected from the group consisting of an extender, an antibiotic, a buffer, an energy source, an antioxidant, a protein source, and a combination thereof. Inseminates according to the present disclosure may be frozen or thawed.

The present disclosure further provides for, and includes, Bos taurus inseminates comprising a sperm having a genome comprising an Animal Haploid Genotype, the inseminate further comprising one or more extenders. The term “extender” refers to any medium that preserves sperm viability. The term “extension” refers to the dilution of sperm and cryoprotectant (e.g., an inseminate) with extender. An extender suitable for use in the selected sperm sample includes a physiologically acceptable carrier. The physiologically acceptable carrier is typically aqueous, and, in certain aspects, includes deionized water. Suitable extenders commonly comprise one or more of the following additional components: a component that maintains osmolality and buffers pH, an organic substance that prevents cold shock and preserves fertility of sperm, a detergent that acts synergistically with certain organic substances to enhance preservation of sperm, an energy source that can be readily utilized by sperm, an antioxidant, which protects sperm from cold shock, a substance that facilitates sperm capacitation, and one or more antibiotics. In aspects according to the present disclosure, the extender may be a commercial extender, such as BOVIPRO® CRYOGUARD® (MOFA Global, Wisconsin, USA), ANDROMED® (Minitube, Verona, Wis.), ANDROMED® CSS (Minitube, Verona, Wis.), TRILADYL® (Minitube, Verona, Wis.), BILADYL® (Minitube, Verona, Wis.), STERIDYL® (Minitube, Verona, Wis.), and BIOCIPHOS (IMV, France). See for example U.S. Patent Publication No. 2003/0157475 published Aug. 21, 2003.

The present disclosure further provides for, and includes, Bos taurus inseminates comprising at least one sperm having a genome comprising an Animal Haploid Genotype and further comprising extenders that are a source of protein. Suitable protein sources include, but are not limited to egg yolk, milk, BSA, and derivatives and combinations thereof. Inseminates comprising a protein source may further include one or more additional components selected from the group consisting of osmolytes, a buffer, and organic substances that prevent cold shock, detergents, energy sources, antioxidants, one or more antibiotics, and a combination thereof.

The present disclosure further provides for, and includes, Bos taurus inseminates comprising a sperm having a genome comprising an Animal Haploid Genotype wherein the osmolality of the inseminate is controlled. The term “osmolality,” as used herein, is a measure of the osmotic pressure of dissolved solute particles in an aqueous solution (e.g., an extender). The solute particles include both ions and non-ionized molecules. Osmolality is expressed as the concentration of osmotically active particles (i.e., osmoles) dissolved in 1 kg of water.

Substances helpful in maintaining osmolality and pH within these ranges are well known in the art and can be added to the extender as a solid or already in solution. A buffer containing a salt, a carbohydrate, or a combination thereof can be employed for this purpose. In aspects according to the present disclosure, the osmolality is between about 250 milliosmoles (mOsM) to about 350 mOsM. In certain aspects, the pH of the Bos taurus inseminate is between 6.9 and 7.5. Specific examples of osmolytes and buffers include sodium citrate, Tris[hydroxymethyl]aminomethane, and TES (N-Tris [Hydroxymethyl]methyl-2-aminoethanesulfonic acid), and monosodium glutamate buffers; milk; HEPES-buffered medium; and any combination thereof. The component employed to help maintain osmolality and provide buffering capacity in a particular application can vary depending on the other components of the extender and, in some cases, on the species from which the sperm are derived. The selection of such a component for use in the present teachings is, however, within the level of skill in the art. Inseminates comprising an osmolyte may further include one or more additional components selected from the group consisting of a protein source, a buffer, and organic substances that prevent cold shock, detergents, energy sources, antioxidants, and one or more antibiotics.

The present disclosure further provides for, and includes, Bos taurus inseminates comprising a sperm having a genome comprising an Animal Haploid Genotype; wherein the inseminate further comprises extenders that comprise an antibiotic, since substantial bacterial growth can threaten sperm viability and increase the risk of infection of the host in artificial insemination or in vitro fertilization procedures. Any of a variety of antibiotics useful in the cryopreservation of cells can also be employed in the extender. The selection of a suitable antibiotic depends on the species from which the sperm was obtained, the procedures involved in obtaining and handling the sperm sample, and the specific microorganism(s) to be targeted. Exemplary antibiotics include tylosin, gentamicin, lincomycin, spectinomycin, linco-spectin (a combination of lincomycin and spectinomycin), penicillin, streptomycin, and ticarcillin, which can be used alone or in combination. However, one skilled in the art can readily determine other antibiotics suitable for use in the extender. Inseminates comprising an osmolyte may further include one or more additional components selected from the group consisting of a protein source, a buffer, and organic substances that prevent cold shock, detergents, energy sources, antioxidants, and osmolytes.

The present disclosure provides for and includes Bos taurus inseminates comprising a sperm having a genome comprising an Animal Haploid Genotype and; wherein the inseminate further comprises organic stress reducing agents. Organic stress reducing agents (OSR) provide improved motility, viability, fertility, and integrity of sperm cells in the Bos taurus inseminates of the present disclosure. Suitable OSRs include, but are not limited to catalase, superoxide dismutase (SOD), a SOD mimic, glutathione, glutathione reductase, glutathione peroxidase, pyruvate, mercaptoethanol, butylated hydroxytoluene (BHT), lipoic acid, flavins, quinines, vitamin K (and related vitamers), vitamin B12 (and related vitamers), vitamin E (and related vitamers), tocopherols, tocotrienols, α-tocopheryl, alpha ketoglutarate (AKG), malondialdehyde (MDA), asymmetric dimethylarginine (ADMA) and biologically active derivatives thereof. Bos taurus inseminates and compositions according to the present disclosure can include one or more OSRs. Inseminates comprising a one or more OSRs may further include one or more additional components selected from the group consisting of a protein source, a buffer, and organic substances that prevent cold shock, detergents, energy sources, antioxidants, and osmolytes.

The present specification provides for, and includes, inseminates comprising a sperm having a genome comprising an Animal Haploid Genotype. In an aspect, the inseminate comprises Bos taurus sperm comprising at least 90% of each of the recited loci. In an aspect, the inseminate comprises at least 95% of the recited loci. In an aspect, the inseminate comprises at least 97% the recited loci. In an aspect, the inseminate comprises at least 98% of the recited loci. In an aspect, the inseminate comprises at least 99% of the recited loci. In an aspect, the inseminate comprises at least 99.5% of the recited loci. In an aspect, the inseminate comprises sperm cells from Bos taurus Animal, wherein a sample of cells of Animal comprises the Deposit. As provided herein, the inseminate may be fresh, frozen, or frozen and thawed and may further comprise a component selected from the group consisting of an extender, an antibiotic, a buffer, an energy source, an antioxidant, a protein source, and a combination thereof.

The present specification provides for, and includes, inseminates wherein the sperm have undergone one or more selections to prepare selected sperm inseminate. In some aspects, selected sperm inseminates may be selected based on sex-type. In some aspects, the sperm are sex selected prior to preparing the selected sperm inseminate. In various aspects, the inseminate is prepared and then subjected to a sex-type selection processes. In various aspects, the sex-type selection is performed prior to freezing the inseminate. In various aspects, the sex-type selection process is performed after thawing of a frozen inseminate.

Sex-type selection may be performed based on slight differences in the physical characteristics of sperm cells. Selection may also be performed based on nucleic acid sequence. A variety of methods are available for selecting cells including flow-cytometric methods. Importantly, the selection and subsequent processing of sperm creates challenges to maintain fertility and successful insemination when used in breeding. Methods of sex-type selection are known in the art.

The present disclosure provides for, and includes, sex selected inseminates comprising sperm having a genome comprising an Animal Haploid Genotype.

In an aspect, the sex selected inseminate comprises Bos taurus sperm comprising at least 90% of the recited loci. In an aspect, the sex selected inseminate comprises at least 95% of the recited loci. In another aspect, the sex selected inseminate comprises at least 97% of the recited loci. Other aspects provide sex selected inseminates comprising at least 98% of the recited loci. In an aspect, the sex selected inseminate comprises at least 99% of the recited loci. In a further aspect, the sex selected inseminate comprises at least 99.5% of the recited loci. In an aspect, the sex selected inseminate comprises sperm cells from Bos taurus Animal, wherein a sample of cells of Animal comprise the Deposit. As provided herein, the sex selected inseminate may be fresh, frozen, or frozen and thawed and may further comprise one or more components selected from the group consisting of an extender, an antibiotic, a buffer, an energy source, an antioxidant, and a protein source. As provided herein, the sex selected inseminate may be skewed toward X-chromosome bearing or Y-chromosome bearing populations of spermatozoa. In an aspect, the sex selected inseminate comprises 10⁴ or more, 10⁵ or more, or 10⁶ or more cells. In an aspect, the sex selected inseminate is provided in a container and comprises between 10⁴ and 10⁷ cells. In an aspect, the number of sex selected cells in the container is between 10⁵ and 10⁷ cells.

The present disclosure provides for, and includes, a container of a Bos taurus inseminate comprising a plurality of sperm cells having attributes of Bos taurus semen Animal, wherein a sample of cells of Animal comprises the Deposit.

The present disclosure provides for, and includes oocytes extracted from a cow or heifer comprising an Animal F1 germplasm. An average collection for IVF can result in an average of about 20 oocytes. The present disclosure further provides for treating the oocytes with TCM-99 (Sreenivas, D. 2013, J. Aller. Ther., 4(2)), or other oocyte maturation media known in the art. TC 199 stock solution is commercially available from Minitube (Verona, Wis.), and other vendors. The present disclosure further provides for and includes methods of capacitation of sperm known in the art (see, for example, Parrish, J J., 2014, Theriogenology, 81(1), 67-73). The present disclosure further provides for and includes methods of in vitro fertilization known in the art for fertilizing mature ova of Animal's F1 offspring.

The present disclosure provides for and includes methods of Embryo Transfer to increase the number of possible offspring for daughters of Animal. The present disclosure provides for and includes superovulation of Animal's daughters as described in Example 6. The present disclosure further provides for the released ova being fertilized by either artificial insemination or natural service.

The present disclosure provides for, and includes, the distinguishing attributes of Animal and the inseminates of the present disclosure in Table 2. The unexpected and desirable combination of attributes is particularly valuable for dairy herd improvement and breeding purposes. In the following tables, “T” means that the animal tested free of the associated genotype and “0” means there is no chance that the animal is a carrier. All values presented herein were current as of the April 2020 Sire Summary.

TABLE 2
Composite scores, linear trait scores and genomic results for Animal

| | | |
|---|---|---|
| | Sex | M |
| | id17 | HO840003210132823 |
| | Birth Date | 2019 Aug. 9 |
| Net Merit Score | NM$ | 928 |
| productive life | PL | 5.4 |
| somatic cell score | SCS | 2.79 |
| Daughter Pregnancy Rate | DPR | −0.4 |
| Cow Livability | LIV | 2.7 |
| PTA MILK | Milk | 1462 |
| PTAF | Fat | 122 |
| PTAP | Pro | 54 |
| Heifer Conception Rate | HCR | −0.5 |
| Cow Conception Rate | CCR | 0.7 |
| Stature | STA | −0.44 |
| Strength | STR | −0.5 |
| Body Depth | BDE | −0.36 |
| Daily Form | DFM | 1.11 |
| Rump Angle | RPA | −1.1 |
| Thurl Width | TRW | 0.15 |
| Rear Legs Side View | RLS | 0.25 |
| Rear Legs Rear View | RLR | 0.8 |
| Fore Udder Height | FTA | 0.12 |
| PTA Type | PTAT | 0.89 |
| Fore Udder Attachment | FUA | 0.93 |
| Rear Udder Height | RUH | 2.09 |
| Rear Udder Width | RUW | 1.92 |
| Udder Cleft | UCL | 0.49 |
| Udder Depth | UDP | 0.13 |
| Front Teat Placement | FTP | 0.6 |
| Teat Length | TLG | −0.62 |
| Feet Leg Score | FLS | 0.53 |
| Rear Teat Placement | RTP | 0.78 |
| Sire Calving Ease | SCE | 6.7 |
| Service Sire Stillbirth | SSB | 8.1 |
| Daughter Stillbirth | DSB | 5.8 |
| Daughter Calving Ease | DCE | 5.6 |
| Udder Composite Index | UDC | 1.37 |
| Feet & Legs Composite | FLC | 0.71 |
| Total Performance Index ® | TPI ® | 3032 |
| Brachyspina/FANCI | HH0 | T |
| APAF1 | HH1 | T |
| Unknown Lethal | HH2 | T |
| SMC2 | HH3 | T |
| GART | HH4 | T |
| TFB1M | HH5 | T |
| BLAD/ITGB2 | HHB | T |
| CVM/SLC35A3 | HHC | T |
| Cholesterol deficiency/APOB | HCD | 0 |
| Mulefoot/LRP4 | HHM | T |
| red color/MC1R | HHR | T |
| Polledness/POLLED | HHP | T |
| Black/Red Coat Color | HBR | T |
| Dominant Red Coat Color | HDR | T |
| | naab_code | 029HO19496 |
| | Dam ID | HO840003143328234 |
| | Sire ID | HO840003141494670 |
| Maternal Grand Sire | MGS ID | HO840003128557482 |
| Hypocalcemia | Milk Fever | 0.1 |
| Dispaced Abomasum | Dab | 0.4 |
| | Ketosis | 1.2 |
| | Mastitis | 0.5 |
| | Metritis | 1.1 |
| | Retained | 0 |
| | Placenta | |

The present disclosure provides for, and includes, F1 progeny animals, and parts thereof comprising a diploid or haploid genome wherein the diploid genome comprises 90% to 100% of loci characterizing the Animal Homozygous Genotype, and said F1 haploid genome having at least 50% of the loci characterizing the Animal Homozygous Genotype. F1 progeny animals are obtained by cloning a first parent animal prepared by somatic cell nuclear transfer or breeding of the Bos taurus cells of the present application with a second parent.

F1 progeny animals obtained from crosses of Bos taurus animals having the Animal Genotype of the present application with elite DM animals (or SM animals for female germplasm) are likely to retain many loci in their homozygous state. Moreover, as nearly all elite DM (and SM) animals are genotyped, a breeder can predict prior to the cross whether progeny will share the desired loci and estimate the expected number of homozygous loci. In most aspects, these valuable F1 progeny are subsequently screened and the most desirable elite F1 progeny identified and selected for maintaining the SM (or DM herd) and for further improvement. Thus, breeding strategies and decisions, informed by genomic data, can be made to continuously improve the germplasm and further, to lock in trait combinations by fixing homozygous alleles. Analysis and selection are necessary to not only make further improvements in breeding stock, but to maintain existing breeding stock quality. The animals of the present disclosure and those bred and selected from them do not occur naturally. Rather, under natural conditions, populations revert towards heterogeneity. See Park et al., Genome Biology 16: 234 (2015). Feral cattle (i.e., cattle not selected in breeding programs) are genetically distinct. See MacNeil et al., Animal Genetics 38:193-197 (2007).

Thus, the present disclosure provides for, and includes, methods of breeding and selection to retain the homozygous alleles of Animal Genotype and generate new homozygous alleles from among the heterozygous loci of the Animal Heterozygous Genotype. In select crosses with elite second parents (e.g., a DM or SM second parent), selection of preferred traits comprising at least 40% of homozygous loci characterizing the Animal Homozygous Genotype. Further, for an F1 progeny of Animal, the probability of any individual heterozygous locus becoming homozygous is 50% when crossed with a second parent that is homozygous at a locus. When the second parent is heterozygous at a locus, there is a 25% probability of an offspring being homozygous for a desired allele. Thus, over time, the total number of homozygous loci will increase in select crosses.

Also provided for, and included in, the present disclosure, are F1 progeny animals that have desirable EBV or GEBV scores. Such animals are important Sires of Females (SF) that are useful for breeding commercial herds and populations. Like breeding F1 progeny for the production of DM and SM animals, the second parent can be a select parent or an elite parent. Select parents for the production of F1 progeny animals that are either SF or DF animals have positive EBV scores for one or more PTA traits selected from the group consisting of productive life (PL), somatic cell score (SCS), Daughter Pregnancy Rate (DPR), PTA MILK (Milk), PTAF (Fat), and PTAP (Pro).

As used herein, the term “Animal F1 Diploid Genotype” provides for, and includes, a diploid F1 Bos taurus cells or a plurality of diploid Bos taurus cells comprising improved germplasm characterized by a genome having 90% to 100% of the loci characterizing the Animal Homozygous Genotype. In an aspect, an Animal F1 Diploid Genotype comprises at least 90% of the loci characterizing the Animal Homozygous Genotype. In another aspect, an Animal F1 Diploid Genotype comprises at least 95% of the loci characterizing Animal Homozygous Genotype. In other aspects, an Animal F1 Diploid Genotype comprises at least 97% of the loci of the Animal Homozygous Genotype. In a further aspect, an Animal F1 Diploid Genotype comprises at least 97% of the loci of the Animal Homozygous Genotype. In a further aspect, an Animal F1 Diploid Genotype comprises at least 98% of the loci characterizing the Animal Homozygous Genotype. In another aspect, an Animal F1 Diploid Genotype comprising at least 99% of the loci characterizing the Animal Homozygous Genotype. In another aspect, an Animal F1 Diploid Genotype comprises at least 99.5% of the loci characterizing the Animal Homozygous Genotype. In various aspects, an F1 progeny animal of the present disclosure comprises an Animal F1 Diploid Genotype that comprises the nucleic acid sequences of the Animal Homozygous Genotype. In an aspect, the F1 progeny animal, or part thereof, is an F1 progeny animal of Bos taurus Animal, wherein a sample of cells of Animal comprises the Deposit. As provided herein, the present specification provides for parts of F1 progeny animals including cells, cell cultures, and tissues. Also provided are frozen samples of parts of F1 progeny animals. Cells of F1 progeny animals include somatic cells and haploid germ cells (e.g., sperm and ova depending on the sex). In certain aspects, the Bos taurus cells of an F1 progeny animal are non-reproductive cells.

The present disclosure further provides for, and includes, F1 progeny animals, or parts thereof, comprising a diploid genome comprising between 40% and 80% of the homozygous loci characterizing the Animal Homozygous Genotype. In another aspect, the F1 progeny comprise at least 50% of the homozygous loci characterizing the Animal Homozygous Genotype. In another aspect, the F1 progeny comprise at least 60% of the homozygous loci characterizing the Animal Homozygous Genotype. In some crosses, the F1 progeny comprise at least 70% of the homozygous loci characterizing the Animal Homozygous Genotype. In an aspect, F1 progeny comprise at least 80% of the homozygous loci characterizing the Animal Homozygous Genotype. In certain aspects, F1 progeny comprise between 60 and 80% of loci of the Animal Homozygous Genotype that are homozygous. In certain aspects, an animal having an Animal Genotype is crossed to a DM or SM animal, wherein an F1 Animal arising from this cross comprises a genome having at least 90% of the loci characterizing the Animal Homozygous Genotype, and at least 45% of said loci are homozygous. In certain aspects, an animal having an Animal Genotype is crossed to a DM or SM animal, wherein the F1 Animal arising from this cross comprises a genome having at least 95% of the loci characterizing the Animal Homozygous Genotype, and at least 55% of said loci are homozygous. In various aspects, an animal having an Animal Genotype is crossed to a DM or SM animal, wherein the F1 Animal arising from this cross comprises a genome having at least 95% of the loci characterizing the Animal Homozygous Genotype, and at least 65% of said loci are homozygous.

The present disclosure further provides for, and includes, F1 progeny that are F1 hybrid animals. As used herein, an F1 hybrid animal is an animal comprising an Animal Haploid Genotype and a haploid genome of an animal of a different cattle breed. In an aspect, the second parent of the hybrid is a Bos indicus breed. In an aspect, the second parent is a member of a breed selected from the group consisting of Angus (ANG), Beef Shorthorn (SHR), Belgian Blue (BBL), Belted Galloway (BGA), Brahman (BRM), British Shorthorn (BSHN), Brown Swiss (BSW), Dutch Belted (DBE), Dutch Friesian (DFR), East Anatolian Red (EAR), English Longhorn (ELO), Finnish Ayrshire (FAY), French Brown Swiss (BRU), Galloway (GAL), Gascon (GAS), Guernsey (GNS), Hereford (HFD), Jersey (JER), Limousin (LMS), Longhorn (LHR), Milking Shorthorn (MSH), Normande (NOR), Norwegian Red (NRC), Red Angus (RGU), Texas Longhorn (TXL), Wagyu (WAG), and combinations of each.

In aspects according to the present disclosure, included are gametes obtained from F1 progeny of a first parent comprising a diploid genome comprising an Animal Genotype and a second parent having a Net Merit index (NM$) of at least 1000 or a TOTAL PERFORMANCE INDEX® (TPI®) of at least 2000. In an aspect, the second parent comprises a NM$ of at least 800. 400. In a further aspect, the second parent comprises a NM$ of at least 900. Crosses of Animal Genotype animals to improve select DM and SM animals, results in superior F1 animals having an Animal F1 Diploid Genotype. Improved F1 progeny having an Animal F1 Genotype that are produced from select DM animals having loci of the same allele as the Animal Homozygous Genotype. Accordingly, in addition to the at least 90% of the loci characterizing the Animal Homozygous Genotype provided by Animal, a DM animal provides loci comprising nucleic acids having the same allele as the Animal Homozygous Genotype provides for the generation of homozygous loci. In an aspect, the number of homozygous loci thus generated is at least 25%.

As used herein, the term “Animal F1 Haploid Genotype” provides for, and includes, haploid Bos taurus cells or a plurality of haploid Bos taurus cells comprising improved germplasm characterized by a genome comprising between 25% and 100% of the loci selected from the alleles of the Animal Homozygous Genotype. In aspects, an Animal F1 Haploid Genotype comprises between 25% and 50% of the loci selected from the alleles of the Animal Homozygous Genotype. In aspects, the genome comprises at least 30% of the selected loci. In some aspects, the F1 haploid genotype comprises between 25 and 35% of the alleles of the Animal Homozygous Genotype.

In an aspect, the gametes of an F1 progeny animal of the present disclosure comprises an Animal F1 Haploid Genotype having at least 45% of the loci characterizing the Animal Homozygous Genotype. In another aspect, the gamete of an F1 progeny animal of the present disclosure comprises an Animal F1 Haploid Genotype of at least 50% of the loci characterizing the Animal Homozygous Genotype. In yet another aspect, the gamete of an F1 progeny animal of the present disclosure comprises an Animal F1 Haploid Genotype of at least 55% of the loci characterizing the Animal Homozygous Genotype.

As provided herein, gametes of an F1 progeny animal comprise between 55% and 95% of the loci of an Animal F1 Haploid Genotype which in turn comprises the loci characterizing the Animal Homozygous Genotype. As will be understood by persons of skill in the art, the smaller the phylogenetic distance between the first parent and second parent, the greater the probability of obtaining progeny having desirable alleles at a large number of important loci. As discussed, given the availability of genomic data from a second parent, the expectation of obtaining F1 progeny that have a high number of homozygous loci can be determined and accordingly the probability of gametes having desired loci determined. In an aspect, gametes of an F1 progeny animal comprise 60% of the loci of the Animal F1 Haploid Genotype. In another aspect, gametes of an F1 progeny animal comprise 70% of the loci of the Animal F1 Haploid Genotype. In yet another aspect, gametes of an F1 progeny animal comprise 75% of the loci of the Animal F1 Haploid Genotype. Other aspects provide gametes of an F1 progeny animal that comprise 80% of the loci of the Animal F1 Haploid Genotype.

The present disclosure provides for, and includes, methods for improving bovine herds by selective breeding. Generally, selective breeding includes mating through natural service, artificial insemination, in vitro fertilization, and embryo transfer. As provided herein, selective breeding comprises crossing a bull of variety Animal, or a frozen inseminate thereof, to a second parent, by artificially inseminating or inseminating by natural service, and calving a progeny calf. In an aspect, the selective breeding comprises providing an inseminate comprising a sperm having a genome comprising an Animal Haploid Genotype. In another aspect, the disclosure provides for, and includes, providing an F1 inseminate comprising a sperm having a genome comprising an Animal F1 Haploid Genotype. In certain aspects, the second parent is an animal of a herd in need of improvement. In an aspect, a herd in need of improvement is a commercial herd.

In aspects according to the present disclosure, methods of improving herds further includes testing a second parent for the haplotypes HHB, HHC, HHD, HHM, HBR, and HDR and selecting a second parent lacking one or more haplotypes HHB, HHC, HHD, HHM, HBR, or HDR.

The present disclosure further provides for, and includes, a composition comprising a Bos taurus genome comprising an Animal Genotype as recited above. In some configurations, the present teachings include a Bos taurus genome further comprising 90% of the heterozygous loci characterizing the Animal Heterozygous Genotype. In various configurations, a composition of the present teachings comprises an isolated nucleus or plurality thereof, a cell or plurality thereof, or isolated genomic DNA. In some aspects, the composition comprises a Bos taurus genome from a non-reproductive cell.

In some embodiments, the present disclosure provides for and includes, a composition comprising a Bos taurus genome of Animal Genotype. In another aspect, a composition comprises an Animal Haploid Genotype. In a further aspect, a composition comprises an Animal F1 Diploid Genotype. In yet another aspect, a composition comprises an Animal F1 Haploid Genotype. In an aspect, the composition comprises a Bos taurus genome of Bos taurus Animal, wherein a sample of cells of Animal comprises the Deposit.

The present disclosure provides for and includes containers of Bos taurus inseminate comprising a plurality of sperm cells, wherein said plurality of sperm cells have the attributes of Bos taurus Animal, wherein a sample of cells of Animal comprises the Deposit. The present specification provides for and includes containers of inseminates comprising a sperm having a genome comprising an Animal Haploid Genotype. In a further aspect, the containers of inseminate comprise Bos taurus sperm comprising at least 90% of the recited Animal Haploid Genotype loci. In a further aspect, each of the containers of inseminate comprises at least 95% of the recited Animal Haploid Genotype loci. In an aspect, each of the containers of inseminate comprises at least 97% of the recited Animal Haploid Genotype loci. In an aspect, each of the containers of inseminate comprises at least 98% of the recited Animal Haploid Genotype loci. In an aspect, each of the containers of inseminate comprises at least 99% of the recited Animal Haploid Genotype loci. In an aspect, each of the inseminate comprises at least 99.5% of the recited Animal Haploid Genotype loci. In an aspect, the containers of inseminate comprises sperm cells from Bos taurus Animal, wherein a sample of cells of Animal comprises the Deposit. As provided herein, the inseminate in the containers may be fresh, frozen, or frozen and thawed and may further comprise a component selected from the group consisting of an extender, an antibiotic, a buffer, an energy source, an antioxidant, and a protein source.

Also included are containers of Holstein inseminates comprising a sperm having an Animal Haploid Genotype as recited above wherein the sperm of the inseminate is obtained from a Holstein bull having a NM$ of about 900 to about 1500. In an aspect, the sperm of the inseminate is obtained from a Holstein bull having a NM$ of about 1000 to about 1450. In an aspect, the sperm of the inseminate is obtained from a Holstein bull having a NM$ of about 1050 to about 1400. In an aspect, the sperm of the inseminate is obtained from a Holstein bull having a NM$ of about 1100 to about 1350. In an aspect, the sperm of the inseminate is obtained from a Holstein bull having a NM$ of about 1150 to about 1300. In an aspect, the sperm of the inseminate is obtained from a Holstein bull having a NM$ of about 1000. In various aspects, the sperm of the inseminate is obtained from a Holstein bull having a NM$ of about 950, about 1000, about 1050, about 1100, about 1150, about 1200, about 1250, about 1300, about 1350, about 1400, about 1450, or about 1500.

Also included are containers of inseminates comprising a sperm having an Animal Haploid Genotype as recited above wherein the sperm of the inseminate is obtained from a Holstein bull having a TPI® of about 2100 to about 3000. In an aspect, the sperm of the inseminate is obtained from a Holstein bull having a TPI® greater than 2100. In various aspects, the sperm of the inseminate is obtained from a Holstein bull having a TPI® of greater than 2200. In various aspects, the sperm in a container of inseminate is obtained from a Holstein bull having a TPI® of greater than 2300. In another aspect, the sperm of the inseminate is obtained from a Holstein bull having a TPI® greater than 2400. In various aspects, the sperm of the inseminate is obtained from a Holstein bull having a TPI® greater than 2500. In various aspects, the sperm of the inseminate is obtained from a Holstein bull having a TPI® greater than 2600. In various aspects, the sperm of the inseminate is obtained from a Holstein bull having a TPI® greater than 2700. In various aspects, the sperm of the inseminate is obtained from a Holstein bull having a TPI′ greater than 2800. In various aspects, the sperm of the inseminate is obtained from a Holstein bull having a TPI® greater than 2900.

In certain aspects, the present disclosure provides for methods of creating elite F1 Holstein progeny. In various aspects, a second Holstein parent is selected that comprises NM$ of at least 1000, a TPI® of at least 2000 in any given progeny generation. In aspects of the present disclosure, the F1 progeny animals comprise a NM$ of at least 1000 or a TPI® of at least 2000.

The present disclosure further provides for, and includes, methods to prepare improved F1 progeny Holstein animals comprising selecting a second Holstein parent having a NM$ of at least 1000 or a TPI® of at least 2000. In an aspect, the second parent comprises a NM$ of at least 800. In a further aspect, the second parent comprises a NM$ of at least 900.

The present disclosure further provides for, and includes, Holstein F1 progeny animals, or parts thereof, comprising a NM$ of at least 1000 or a TPI® of at least 2000. In an aspect, the Holstein F1 progeny animals, or parts thereof, comprise a NM$ of at least 800. In a further aspect, the F1 progeny animals, or parts thereof, comprise a NM$ of at least 900.

The present disclosure provides for, and includes, processes for storing spermatozoa comprising obtaining a Bos taurus ejaculate spermatozoa, each spermatozoa having an Animal Haploid Genotype as recited above, mixing the Bos taurus ejaculate with an antibiotic to form a sperm dispersion. In an aspect, the Bos taurus ejaculate is an ejaculate of a bull of Bos taurus Animal, wherein a sample of cells of Animal comprises the Deposit. In another aspect, the spermatozoa comprise an Animal F1 Haploid Genotype as recited above. In another aspect, the spermatozoa comprise an Animal F1 Haploid Genotype comprising at least 55% of the loci characterizing the Animal Homozygous Genotype. In another aspect, the spermatozoa comprise an Animal F1 Haploid Genotype comprising at least 60% of the loci characterizing the Animal Homozygous Genotype. In yet another aspect, the spermatozoa comprise an Animal F1 Haploid Genotype comprising at least 65% of the loci characterizing the Animal Homozygous Genotype. In yet another aspect, the spermatozoa comprise an Animal F1 Haploid Genotype comprising at least 70% of the loci characterizing the Animal Homozygous Genotype. In other aspects, the spermatozoa comprise an Animal F1 Haploid Genotype comprising at least 75% of the loci characterizing the Animal Homozygous Genotype. Other aspects provide for spermatozoa comprising an Animal F1 Haploid Genotype comprising at least 80% of the loci characterizing the Animal Homozygous Genotype. Further aspects provide for spermatozoa comprising an Animal F1 Haploid Genotype comprising at least 85% of the loci characterizing the Animal Homozygous Genotype. In addition, the disclosure provides spermatozoa comprising an Animal F1 Haploid Genotype comprising at least 90% of the loci characterizing the Animal Homozygous Genotype. Other aspects provide for obtaining a Bos taurus ejaculate comprising spermatozoa comprising an Animal F1 Haploid Genotype comprising at least 95% of the loci characterizing the Animal Homozygous Genotype. Further aspects provide for spermatozoa comprising an Animal F1 Haploid Genotype comprising at least 99% of the loci characterizing the Animal Homozygous Genotype. As provided herein, the sperm dispersions may further comprise one or more components selected from the group consisting of a cryoprotectant, an extender, an antibiotic, a buffer, an energy source, an antioxidant, and a protein source. In aspects of the present disclosure, the number of cells in a sperm dispersion comprise 1000 or more, 10⁴ or more, 10⁵ or more, or 10⁶ or more cells. In an aspect, the cells are in a container and comprise between 10⁴ and 10⁷ cells. In an aspect, the number of cells is between 10⁵ and 10⁷ cells.

The process for storing spermatozoa further includes preparing a container having a volume of sperm dispersion and freezing the sperm dispersion container prior to storing. As used herein, a “container” is any suitable container, including a straw or sheath. In an aspect, the volume of sperm dispersion is 0.25 milliliters (ml), 0.50 ml, or any volume between 0.1 and 1 ml. As provided by the present specification, the storing comprises maintaining the sperm dispersion at a temperature of −196° C. Also included, and provided for, in the process for storing spermatozoa is sorting said spermatozoa into X-chromosome or Y-chromosome enriched inseminate. In certain aspects, the process may further comprise removing immotile spermatozoa. In aspects of the present disclosure, the number of cells in a straw, sheath or container comprises between 10⁶ to 10⁸ cells. In an aspect, the number of cells in a straw, sheath or container comprises between 10⁴ and 10⁷ cells. In an aspect, the number of cells in a straw, sheath or container comprises between 10⁵ and 10⁷ cells. In an aspect, the concentration of spermatozoa in the sperm dispersion is between about 0.04×10⁶ sperm/ml to about 1.2×10⁸ sperm/ml.

The present disclosure provides for, and includes a combination comprising an elongated container for use in the insemination of a Bos taurus dam and a sperm dispersion comprising a plurality of sperm, each sperm comprising a genome comprising an Animal Haploid Genotype or an Animal F1 Haploid Genotype, wherein the sperm dispersion is contained in the elongated container. In an aspect, the concentration of spermatozoa in the sperm dispersion is between about 0.04×10⁶ sperm/ml to about 1.2×10⁸ sperm/ml.

The present disclosure provides for, and includes, methods of inseminating a Bos taurus dam comprising thawing a frozen inseminate comprising a plurality of sperm cells, each sperm cell having a genome comprising an Animal Haploid Genotype or an Animal F1 Haploid Genotype and artificially inseminating a dam with said thawed inseminate to produce an F1 bovine calf or an F2 bovine calf respectively. In an aspect, the method includes providing a plurality of sperm cells that comprise an Animal Haploid Genotype or an Animal F1 Haploid Genotype comprising at least 55% of the loci characterizing the Animal Homozygous Genotype. In another aspect, the method includes providing a plurality of sperm cells that comprise an Animal Haploid Genotype or an Animal F1 Haploid Genotype comprising at least 60% of the loci characterizing the Animal Homozygous Genotype. In yet another aspect, the method includes providing a plurality of sperm cells that comprise an Animal Haploid Genotype or an Animal F1 Haploid Genotype comprising at least 65% of the loci characterizing the Animal Homozygous Genotype. In yet another aspect, the method includes providing a plurality of sperm cells that comprise an Animal Haploid Genotype or an Animal F1 Haploid Genotype comprising at least 70% of the loci characterizing the Animal Homozygous Genotype. In other aspects, the method includes providing a plurality of sperm cells that comprise an Animal Haploid Genotype or an Animal F1 Haploid Genotype comprise at least 75% of the loci characterizing the Animal Homozygous Genotype. Other aspects of the method provide for sperm cells comprising an Animal Haploid Genotype or an Animal F1 Haploid Genotype comprising at least 80% of the loci characterizing the Animal Homozygous Genotype. Further aspects provide sperm cells comprising an Animal Haploid Genotype or an Animal F1 Haploid Genotype comprising at least 85% of the loci characterizing the Animal Homozygous Genotype. In addition, the methods provide sperm cells comprising an Animal Haploid Genotype or an Animal F1 Haploid Genotype comprising at least 90% of the loci characterizing the Animal Homozygous Genotype. Other aspects provide a frozen inseminate prepared from a Bos taurus ejaculate comprising spermatozoa comprising an Animal Haploid Genotype or an Animal F1 Haploid Genotype comprising at least 95% of the loci characterizing the Animal Homozygous Genotype. Further aspects provide for sperm cells comprising an Animal Haploid Genotype or an Animal F1 Haploid Genotype comprising at least 99% of the loci characterizing the Animal Homozygous Genotype. As provided herein, the inseminate suitable for the methods of insemination may further comprise one or more components selected from the group consisting of a cryoprotectant, an extender, an antibiotic, a buffer, an energy source, an antioxidant, and a protein source.

The present disclosure provides for, and includes, methods for preparing transgenic animals. Also provided for, and included, are transgenic animals having an Animal Genotype and an Animal F1 Diploid Genotype as recited above. Also included, and provided for, are haploid cells comprising a transgene comprising an Animal Haploid Genotype and an Animal F1 Haploid genotype as recited above.

As used herein, “transgenic animal, cell or tissue” includes reference to animals which comprise within their genomes a gene encoding a heterologous polynucleotide. Generally, the gene is stably integrated within the genome such that the expression of the polynucleotide is passed on to successive generations. The gene may be integrated into the genome alone or as part of a recombinant expression cassette. “Transgenic” is used herein to include any cell, cell line, tissue, or organ, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional breeding methods or by naturally occurring events such as random cross-fertilization, non-recombinant viral infection, non-recombinant bacterial transformation, non-recombinant transposition, or spontaneous mutation. The transgene can be introduced into the genomes of the present disclosure directly or through breeding with a transgene containing animal. Methods to prepare transgenic animals are known in the art, for example by pronucleus injection as described by Gordon et al., Proc. Natl. Acad. Sci. USA 77:7380-7384 (1980). Pronucleus injection has been shown to be suitable for large domestic animals including pigs and cattle. See Hammer et al., Nature 315: 680-683 (1985) and Niemann, Proc. Natl. Acad. Sci. USA 101: 7211-7212 (2004). Other methods of generating transgenic animals are known in the art. See, such as and without limitation, Park, et al., Reprod. Fertil. Dev. 26:65-73 (2013).

Also provided for and included, are gene edited animals having an Animal Genotype. Gene edited animals are non-transgenic animals. The animals of the present disclosure can be gene-edited animals having an Animal Genotype, an Animal F1 Diploid Genotype, an Animal Haploid Genotype, or an Animal F1 Haploid Genotype as recited above. Suitable methods for gene editing are known in the art. Methods include, but are not limited to, the methods provided in International Patent Publication No. WO 2015/148761 published Oct. 1, 2015, U.S. Pat. No. 9,868,962, published Jan. 16, 2018, and International Patent Publication No. WO 2017/132239 published Mar. 8, 2017, and references cited therein, all of which are incorporated by reference.

The gene edited animals may contain edited chromosomal sequences. The edited chromosomal sequence may be (1) inactivated, (2) modified, or (3) comprise an integrated sequence resulting in a null mutation. An inactivated chromosomal sequence is altered such that a target protein function as it relates to an undesirable phenotype is impaired, reduced or eliminated. Thus, a genetically edited animal comprising an inactivated chromosomal sequence may be termed a “knock out” or a “conditional knock out.” Similarly, a genetically edited animal comprising an integrated sequence may be termed a “knock in” or a “conditional knock in.” Furthermore, a genetically edited animal comprising a modified chromosomal sequence may comprise a targeted point mutation(s) or other modification such that an altered protein product is produced. Briefly, the process can comprise introducing into an embryo or cell at least one RNA molecule encoding a targeted zinc finger nuclease and, optionally, at least one accessory polynucleotide. The method further comprises incubating the embryo or cell to allow expression of the zinc finger nuclease, wherein a double-stranded break introduced into the targeted chromosomal sequence by the zinc finger nuclease is repaired by an error-prone non-homologous end-joining DNA repair process or a homology-directed DNA repair process. The method of editing chromosomal sequences encoding a protein associated with germline development using targeted zinc finger nuclease technology is rapid, precise, and highly efficient.

Alternatively, the process can comprise using a CRISPR/Cas9 system to modify the genomic sequence. To use Cas9 to modify genomic sequences, the protein can be delivered directly to a cell. Alternatively, an mRNA that encodes Cas9 can be delivered to a cell, or a gene that provides for expression of an mRNA that encodes Cas9 can be delivered to a cell. In addition, either target specific crRNA and a tracrRNA can be delivered directly to a cell or target specific gRNA(s) can be to a cell (these RNAs can alternatively be produced by a gene constructed to express these RNAs). Selection of target sites and designed of crRNA/gRNA are well known in the art.

The animal or cell can be genetically edited using a homing endonuclease. The homing endonuclease can be a naturally occurring endonuclease but is preferably a rationally designed, non-naturally occurring homing endonuclease that has a DNA recognition sequence that has been designed so that the endonuclease targets a chromosomal sequence in a target gene. Thus, the homing endonuclease can be a designed homing endonuclease. The homing endonuclease can comprise, for example, a Clustered Regularly Interspaced Short Palindromic Repeats (CRISPR)/Cas9 system, a Transcription Activator-Like Effector Nuclease (TALEN), a Zinc Finger Nuclease (ZFN), a recombinase fusion protein, a meganuclease, or a combination thereof. The animal or cell is preferably an animal or cell that has been genetically edited using a CRISPR/Cas9 system.

The following are non-limiting exemplary embodiments of the present disclosure:

1. A Bos taurus cell of type Animal, a representative sample of cells of type Animal comprising the Deposit.
2. A frozen vial, a cell culture, a tissue, a zygote, or an embryo comprising a plurality of the Bos taurus cell according to embodiment 1.
3. A bull comprising a plurality of the Bos taurus cell according to embodiment 1.
4. The Bos taurus cell according to embodiment 1, wherein the cell is a sperm cell.
5. Semen comprising a plurality of the Bos taurus cell according to embodiment 4.
6. An embryo produced by contacting the cell according to embodiment 4 with a Bos taurus ovum.
7. A composition of matter comprising the cell according to embodiment 4 and a Bos taurus ovum.
8. A Bos taurus cell produced by somatic cell nuclear transfer of a Bos taurus cell of type Animal, a representative sample of cells of type Animal comprising the Deposit.
9. A frozen vial, a cell culture, a tissue, a zygote, or an embryo comprising a plurality of the Bos taurus cell according to embodiment 8.
10. A bull comprising a plurality of the Bos taurus cell according to embodiment 8.
11. A Bos taurus cell from an F1 offspring of a Bos taurus animal comprising a cell of type Animal, a representative sample of cells of type Animal comprising the Deposit.
12. A frozen vial, a cell culture, a tissue, a zygote, or an embryo comprising a plurality of the Bos taurus cell according to embodiment 11.
13. An animal comprising a plurality of the Bos taurus cell from an F1 offspring according to embodiment 11.
14. A container of semen produced by the animal of embodiment 13, wherein the animal is a bull.
15. The Bos taurus cell from an F1 offspring according to embodiment 11, wherein the F1 offspring is a gene edited animal.
16. The Bos taurus cell from an F1 offspring according to embodiment 11, wherein the cell is an ovum.
17. The Bos taurus cell from an F1 offspring according to embodiment 11, wherein the cell is a sperm cell.
18. Semen comprising a plurality of the Bos taurus cell according to embodiment 17.
19. An embryo produced from a gamete of the animal according to embodiment 13.
20. A composition of matter comprising a gamete from the animal according to embodiment 13 and a gamete cell from a second Bos taurus parent.

### EXAMPLES

### Example 1: Genomic Characterization of Bos taurus Cells

The genotype of Animal was determined using the ILLUMINA® Infinium HTS BeadChip (ILLUMINA®, Inc., San Diego, Calif.) microarray system using the BovineSNP50 v3 BeadChip microarray kit (BeadChip) according to manufacturer's instructions. The BeadChip contained 53,714 highly informative SNP probes uniformly distributed across the entire genome of major cattle breed types at a median spacing of 37.4 kb. The SNP probes were validated in 18 common beef and dairy breeds. The BeadChip is a collaboration of ILLUMINA®, Inc., the USDA-ARS, the University of Missouri, and the University of Alberta. More than 22,000 SNP probes target novel SNP loci found in pooled populations of economically important beef and dairy cattle. The BeadChip is well known and extensively used by those of skill in the art. Other suitable genotyping chips and technologies can be used, including but not limited to the GENESEEK® GENOMIC PROFILER™ High-Density array (GGP HD150K; Neogen Genomics, Lincoln Nebr.) and the NEOGEN® GGP Bovine 50k chip (Neogen Genomics, Lincoln Nebr.) that contain 139376 and 47843 SNPs respectively.

A wide variety of publicly available resources are known in the art including the bovine reference genome (Bovine Genome Sequencing and Analysis Consortium, Science 324(5926):522-8), Btau (available at ftp(dot)hgsc(dot)bcm.tmc.edu/pub/data/Btaurus/fasta), and the Bovine HapMap Consortium data set (Bovine HapMap Consortium, Science 324(5926):528-32 (2009) available on the internet at bovinehapmap(dot)org). Analysis tools and sequence data are maintained by the Bovine Genome Database (BGD) that is supported by the European Union's Seventh Framework Programme for research, technological development and demonstration (Grant Agreement No. 613689), and the USDA National Institute of Food and Agriculture. BGD is hosted at the University of Missouri.

TABLE 3
BovineSNP50 BeadChip Sources

| | BovineSNP50 | BovineSNP50 | BovineSNP50 |
|---|---|---|---|
| Source | v1 Probes | v2 Probes | v3 Probes |
| Novel SNPs ^{a} | 23,840 | 24,181 | 22,299 |
| Bovine HapMap Data | 12,298 | 12,342 | 11,607 |
| Set | | | |
| Btau Assembly SNPs | 9361 | 9404 | 9086 |
| Whole-Genome | 5808 | 6038 | 5485 |
| Shotgun Reads ^{b} | | | |
| Holstein BAC | 1409 | 1411 | 1238 |
| Sequence | | | |
| Parentage | 116 | 120 | 200 |
| Other | 1169 | 1113 | 3384 |
| Total | 54,001 | 54,609 | 53,218 |

^{a} Derived from sequencing common cattle breeds using the ILLUMINA ® GenomeAnalyzer.
^{b} Obtained from six breeds: Norwegian Red, Holstein, Brahman, Angus, Jersey, and Limousin.

### Example 2: Breeding and Selection of Animal

Animal was a progeny animal of a breeding program for the improvement of dairy herds and breeding purposes. Animal had the genotype as provided above in Table 1 and the predicted characteristics as provided above in Table 2. The definitions and abbreviations are provided above. Animal was an individual of the Holstein breed.

Animal was a cross between Sire and Dam. The composite scores and linear trait results of Sire and Dam are provided below in Table 4. Like Animal, Sire and Dam were SM and DM animals respectively. Sire comprised homozygous loci characterizing the Sire Homozygous Genotype and heterozygous loci characterizing the Sire Heterozygous Genotype. Dam comprised homozygous loci characterizing the Dam Homozygous Genotype and heterozygous loci characterizing the Dam Heterozygous Genotype.

Sire was the progeny of Paternal Grand-Sire and Paternal Grand-Dam. Dam was the progeny of Maternal Grand-Sire and Maternal Grand-Dam. The composite scores and linear trait results for Animal's grandparents are provided below.

The genomes, composite traits and other characteristics of the parents and grandparents were determined according to methods known in the art and are presented below in Tables 4 and 5.

TABLE 4
Genomic Report for Parents of Animal

| | Sire | Dam |
|---|---|---|
| Sex | M | F |
| id17 | HO840003141494670 | HO840003143328234 |
| Birth Date | 2017 Sep. 24 | 2017 Oct. 23 |
| NM$ | 832 | 805 |
| PL | 5.8 | 5.6 |
| SCS | 2.81 | 2.84 |
| DPR | 0.9 | −0.6 |
| LIV | 3.6 | 3 |
| Milk | 1225 | 1806 |
| Fat | 101 | 95 |
| Pro | 52 | 53 |
| HCR | 0.2 | 0.9 |
| CCR | 2.3 | 0.1 |
| STA | −0.36 | −0.61 |
| STR | −0.02 | −1.07 |
| BDE | −0.31 | −0.6 |
| DFM | 0.03 | 1.75 |
| RPA | −0.21 | −0.69 |
| TRW | 0.03 | 0.14 |
| RLS | −0.17 | 0.59 |
| RLR | 0.84 | 0.38 |
| FTA | −0.03 | −0.19 |
| PTAT | 0.39 | 0.9 |
| FUA | 0.6 | 0.37 |
| RUH | 0.76 | 1.74 |
| RUW | 0.7 | 1.6 |
| UCL | 0.18 | 0.63 |
| UDP | 0.09 | −0.26 |
| FTP | 0.61 | 0.62 |
| TLG | −0.23 | −0.73 |
| FLS | 0.25 | 0.53 |
| RTP | 0.89 | 1.03 |
| SCE | 7.3 | 6.8 |
| SSB | 8.2 | 7.6 |
| DSB | 5.6 | 6.8 |
| DCE | 5.9 | 6.7 |
| UDC | 0.66 | 1.06 |
| FLC | 0.47 | 0.62 |
| TPI ® | 2918 | 2880 |
| HH0 | T | T |
| HH1 | T | T |
| HH2 | T | T |
| HH3 | T | T |
| HH4 | T | T |
| HH5 | T | T |
| HHB | T | T |
| HHC | T | T |
| HCD | 0 | 0 |
| HHM | T | T |
| HHR | T | T |
| HHP | T | T |
| HBR | T | T |
| HDR | T | T |
| naab_code | 029HO18858 | NULL |
| Dam ID | HOUSA000074258448 | HO840003133318719 |
| Sire ID | HO840003129038181 | HO840003128557482 |
| MGS ID | HOUSA000072128125 | HOUSA000072128216 |
| Milk Fever | 0.2 | 0.1 |
| Dab | 0.4 | 0.2 |
| Ketosis | 0.9 | 1 |
| Mastitis | 1 | 1 |
| Metritis | 1 | 0.5 |
| Retained Placenta | 0.1 | −0.3 |

TABLE 8
Genomic Report for Grandparents of Animal

| | Paternal Grand Sire | Paternal Grand Dam | Maternal Grand Sire | Maternal Grand Dam |
|---|---|---|---|---|
| Sex | M | F | M | F |
| id17 | HO840003129038181 | HO840000074258448 | HO840003128557482 | HO840003133318719 |
| Birth Date | 2015 Jul. 28 | 2015 Nov. 1 | 2015 Nov. 15 | 2015 Nov. 10 |
| NM$ | 705 | 656 | 819 | 592 |
| PL | 5.2 | 5.4 | 5.2 | 5.7 |
| SCS | 2.78 | 2.83 | 2.77 | 2.74 |
| DPR | 1.3 | 0.5 | 0 | 0.2 |
| LIV | 2 | 2.9 | 3.9 | 4 |
| Milk | 1623 | 590 | 513 | 1272 |
| Fat | 77 | 71 | 113 | 51 |
| Pro | 56 | 37 | 33 | 39 |
| HCR | −2.4 | 1.8 | 1.4 | −0.5 |
| CCR | 1.6 | 2.3 | 0.7 | 0.4 |
| STA | −0.33 | −0.68 | −1.08 | −0.19 |
| STR | 0.24 | 0.04 | −0.81 | −0.38 |
| BDE | −0.26 | −0.34 | −1.01 | −0.46 |
| DFM | −0.39 | 0 | −0.07 | 0.55 |
| RPA | 0.01 | −0.88 | 0.01 | −0.62 |
| TRW | 0.33 | 0.35 | −0.22 | −0.34 |
| RLS | −1.73 | 0.8 | −1.17 | 0.12 |
| RLR | 1 | 1.16 | 0.91 | 0.42 |
| FTA | 0.64 | −0.44 | −0.38 | 0.97 |
| PTAT | 0.27 | 0.48 | 0.17 | 0.74 |
| FUA | 0.76 | 0.67 | 0.16 | 0.74 |
| RUH | 0.99 | 0.81 | 0.51 | 1.71 |
| RUW | 0.91 | 0.75 | 0.47 | 1.57 |
| UCL | −0.49 | −0.16 | −0.01 | 0.04 |
| UDP | 0.34 | −0.15 | −0.21 | 0.69 |
| FTP | −0.31 | 0.07 | 0.54 | −0.06 |
| TLG | 0.2 | 0.69 | −0.27 | −0.66 |
| FLS | −0.05 | 0.61 | 0.31 | 0.59 |
| RTP | −0.28 | 0 | 0.74 | −0.23 |
| SCE | 7.6 | 6.5 | 6.4 | 6.9 |
| SSB | 9 | 7 | 6.7 | 7.6 |
| DSB | 5.4 | 4.7 | 5.3 | 6.7 |
| DCE | 5.4 | 5.8 | 6.9 | 7.5 |
| UDC | 0.67 | 0.57 | 0.52 | 1.09 |
| FLC | 0.24 | 0.86 | 0.64 | 0.66 |
| TPI ® | 2808 | 2703 | 2835 | 2648 |
| HH0 | T | T | T | T |
| HH1 | T | T | T | T |
| HH2 | T | T | T | T |
| HH3 | T | T | T | T |
| HH4 | T | T | T | T |
| HH5 | T | T | C | T |
| HHB | T | T | T | T |
| HHC | T | T | T | T |
| HCD | 0 | 0 | 0 | 0 |
| HHM | T | T | T | T |
| HHR | T | T | T | T |
| HHP | T | T | T | T |
| HBR | T | T | T | T |
| HDR | T | T | T | T |
| naab_code | 200HO10729 | NULL | 029HO18296 | |
| Dam ID | HO840003012130340 | HOUSA000072754161 | HO840003013177242 | HOUSA000072606690 |
| Sire ID | HO840003013129308 | HOUSA000072128125 | HOUSA000072254526 | HOUSA000072128216 |
| MGS ID | HOUSA000070625790 | HOUSA000069981349 | HONLD000543756297 | HOUSA000069169951 |
| Milk Fever | 0 | 0.1 | 0.2 | 0.1 |
| Dab | 0.2 | 0.3 | 0.6 | 0.3 |
| Ketosis | 0.6 | 0.8 | 1.7 | 0.5 |
| Mastitis | 1.1 | 1.4 | 0.6 | 2.8 |
| Metritis | 1.1 | 0.3 | 0.8 | 0.2 |
| Retained Placenta | 0.2 | −0.3 | −0.2 | −0.3 |

Notably, selective breeding has increased the Sire NM$ from 832 to 928 in Animal. Dam had a NM$ of 805. As demonstrated in Table 9, other desirable traits are similarly improved.

TABLE 9
Comparison of Genomic Traits

| Trait | Animal | Sire | Dam |
|---|---|---|---|
| NM$ | 928 | 832 | 805 |
| Fat | 122 | 101 | 95 |
| TPI ® | 3032 | 2918 | 2880 |

### Example 3: Culture of Bos taurus Cells

Adult Bos taurus fibroblast cultures are established from ear punch or tail clip from Animal at an age of less than one year according to standard methods.

Briefly, an ear punch or tail clip is treated with collagenase and digested. The collagenase treated cells are then collected by centrifugation and washed with buffered saline. The washed cells are treated with trypsin and triturated and a suspension of cells plated on standard culture dishes and cultured under standard conditions to prepare a primary culture. The cells are cultured and either fed or split 1:4 to 1:6 and the culture expanded. Cells are harvested when the culture reaches at least 10 million. At confluence, cells are collected by trypsin/EDTA treatment and centrifugation. Cells are resuspended and counted and then collected and resuspended at a concentration of 1×10⁶-10⁷/mL. Cells are diluted with freezing medium (90% FBS+10% DMSO). Tubes are transferred to −80° C. overnight before being placed in liquid nitrogen for long term storage. Frozen cells prepared via this method are deposited with the ATCC under Accession No. PTA TBD.

### Example 4: Somatic Cell Nuclear Transfer (SCNT) of Animal Cells

Somatic cell nuclear transfer (SCNT) is performed according the method of Ross and Cibelli, Methods Mol Biol. 636. 155-77 (2010). See also U.S. Pat. No. 6,011,197 issued Jan. 4, 2000, to Strelchenko et al.

Briefly, five to seven days before performing SCNT, a culture of fibroblasts as described in Example 3 are plated in four well dishes at a density of 100,000 cells per well and cultured. The cells are synchronized in the G0 stage by contact inhibition. Oocytes are harvested from either slaughterhouse-derived ovaries or from live animals by ultrasound-guided oocyte aspiration. The oocytes are matured in vitro and enucleated. The cultured fibroblasts are injected into enucleated oocytes and oocyte-cell fusion is induced using a square DC pulse generator. Fused oocytes are activated using ionomycin and cultured under standard conditions. At 48 h after activation, noncleaved embryos are removed from culture and at 72 h after activation, the culture medium is supplemented with serum and cultured for seven days before being recovered and implanted in synchronized recipients. Calves are born normally to the surrogate mother and are genetically identical offspring to Animal. In addition to providing a source of inseminate for herd improvement, new cultures of cells as described in Example 1 can be prepared. The high availability of bovine oocytes and the relatively higher efficiency levels usually obtained in cattle provide for the use of SCNT for both commercial and research purposes.

### Example 5: Breeding of Animal Progeny

Mature bulls prepared by the methods of the present specification are used for breeding purposes using conventional artificial insemination methods. Alternatively, bulls prepared by SCNT can be used for natural service.

Frozen inseminate obtained as provided below in Example 6 is provided for artificial insemination for the improvement of existing herds. Breeding for the improvement of existing herds does not generally require consideration of the recipient heifer or cow. In an aspect, a dam is selected from a herd in need of improvement and artificially inseminated with Animal inseminate. In an aspect, a herd in need of improvement has an average NM$ index of less than 700. In an aspect, a herd in need of improvement has an average NM$ index of between 700 and 1000. After gestation, calves are born and evaluated for production, composite traits, and predicted transmitting abilities. In some cases, genomic testing is performed. The resulting calves have improved NM$ and other desirable traits as compared to the parent dam and as compared to other calves born in the herd.

The frozen inseminate obtained as provided below in Example 6 is also used for the generation of elite bulls and heifers. Frozen inseminate obtained as provided below in Example 6 is provided for artificial insemination to an elite DM animal having an NM$ index of at least 900. After gestation, the calf is genotyped and identified for predicted characteristics, composite traits, and predicted transmitting abilities. A calf having improved traits is selected and used for further breeding and for the improvement of existing herds.

### Example 6: Progeny Generation Using In Vitro Fertilization and Embryo Transfer

Semen from mature Animal bulls is collected by electroejaculation or by other methods known in the art: The collected semen is frozen in straws per methods known in the art. Progeny are generated by thawing a straw of frozen semen and the thawed semen used for artificial insemination. In short, about 5 ml to about 15 ml of semen is collected from a bull after being electroejaculated and mixed with a suitable extender and cryoprotectant. About 10 ml of semen is collected and mixed with about 240 ml of TRILADYL™ solution (Minitube of America, Verona, Wis.) The mixture of semen, extender and cryoprotectant is then placed in plastic straws. Straws containing about 20 million motile sperm in a volume of about ½ ml are obtained and frozen until needed. Prior to use, frozen straws are thawed.

The collected semen of the present specification may be frozen according to standard methods in the art as discussed above.

### Example 7: Generation of Multiple Embryos by Superovulation and In Vitro Fertilization

The Animal inseminate collected as provided in Example 6 is used for in vitro fertilization of oocytes collected from a heifer or cow to create multiple embryos. The heifer or cow is treated with follicle stimulating hormone to induce multiple ovulations. Following superovulation, the donor heifer or cow is bred using artificial insemination of the Animal inseminate. About seven days after insemination, embryos are non-surgically collected by ‘flushing’ from the donor's uterus and transferred into synchronous recipients that serve as surrogate mothers. Embryos may be frozen for implantation at a later date.

Embryos are also generated using IVF collection of unfertilized oocytes from the ovaries of a donor cow or heifer. Oocytes are fertilized in vitro and transferred seven days after fertilization following incubation under controlled conditions. IVF collection and fertilization allows for the generation of multiple embryos obtained from open cows, pregnant cows, heifers and females having difficulty in conventional breeding. IVF collection also provides for collection of oocytes from donors shortly after death.

Embryos are transferred to a surrogate and gestated until birth.

All publications are herein incorporated by reference, each in their entirety.

What is claimed is:

1. A Bos taurus cell of type HO840003210132823, a representative sample of cells of type HO840003210132823 having been deposited under ATCC accession number PTA-TBD.

2. A frozen vial, a cell culture, a tissue, a zygote, or an embryo comprising a plurality of the Bos taurus cell according to claim 1.

3. A bull comprising a plurality of the Bos taurus cell according to claim 1.

4. The Bos taurus cell according to claim 1, wherein the cell is a sperm cell.

5. Semen comprising a plurality of the Bos taurus cell according to claim 4.

6. An embryo produced by contacting the cell according to claim 4 with a Bos taurus ovum.

7. A composition of matter comprising the cell according to claim 4 and a Bos taurus ovum.

8. A Bos taurus cell produced by somatic cell nuclear transfer of a Bos taurus cell of type HO840003210132823, a representative sample of cells of type HO840003210132823 having been deposited under ATCC accession number TBD.

9. A frozen vial, a cell culture, a tissue, a zygote, or an embryo comprising a plurality of the Bos taurus cell according to claim 8.

10. A bull comprising a plurality of the Bos taurus cell according to claim 8.

11. A Bos taurus cell from an F1 offspring of a Bos taurus animal comprising a cell of type HO840003210132823, a representative sample of cells of type HO840003210132823 having been deposited under ATCC accession number TBD.

12. A frozen vial, a cell culture, a tissue, a zygote, or an embryo comprising a plurality of the Bos taurus cell according to claim 11.

13. An animal comprising a plurality of the Bos taurus cell from an F1 offspring according to claim 11.

14. A container of semen produced by the animal of claim 13, wherein the animal is a bull.

15. The Bos taurus cell from an F1 offspring according to claim 11, wherein the F1 offspring is a gene edited animal.

16. The Bos taurus cell from an F1 offspring according to claim 11, wherein the cell is an ovum.

17. The Bos taurus cell from an F1 offspring according to claim 11, wherein the cell is a sperm cell.

18. Semen comprising a plurality of the Bos taurus cell according to claim 17.

19. An embryo produced from a gamete of the animal according to claim 13.

20. A composition of matter comprising a gamete from the animal according to claim 13 and a gamete cell from a second Bos taurus parent.
